(19) United States
(12) Patent Application Publication
(10) Pub. No.: US 2024/0148504 A1
(43) Pub. Date: May 9, 2024

(54) CATHETER DEVICE FOR DELIVERING A MEDICAL IMPLANT ALLOWING RESHEATHING OF THE IMPLANT

(71) Applicant: BIOTRONIK AG, Buelach (CH)

(72) Inventors: Jeff Alan Burke, Fallbrook, CA (US); Peter D'Aquanni, Murrieta, CA (US); Andy Edward Denison, Temecula, CA (US); Christopher Scott Hayden, Winchester, CA (US); Markus Foerster, Auenstein (CH); Michael Walther, Winterthur (CH); Gereon Kindler, Zuerich (CH); Lukas Frey, Langenthal (CH); Ole Blank, Zuerich (CH); Calvin Bahr, Buelach (CH)

(21) Appl. No.: 18/549,982

(22) PCT Filed: Mar. 15, 2022
(86) PCT No.: PCT/EP2022/056681

§ 371 Date: Sep. 11, 2023

Related U.S. Application Data
(60) Provisional application No. 63/312,936, filed on Feb. 23, 2022.
(60) Provisional application No. 63/312,916, filed on Feb. 23, 2022.
(60) Provisional application No. 63/163,164, filed on Mar. 19, 2021.

(30) Foreign Application Priority Data
May 11, 2021 (EP) 21173211.0

(51) Int. Cl. A61F 2/24; A61M 25/01

(52) U.S. Cl. CPC A61F 2/2436; A61M 25/0105

(57) ABSTRACT

A catheter device for implanting a medical implant such as a prosthetic heart valve has a handle configured to control/steer multiple sheaths of the catheter device. A handle includes a grip portion, a rotatable deployment knob and a traveler. An outer sheath is connected to the traveler and the deployment knob is operatively connected to the traveler such that the traveler and thereby the outer sheath are moved along the longitudinal axis with respect to the inner sheath. The handle includes a rotatable axial positioning knob and a rotatable deflection knob. The deployment knob is arranged proximally to the axial positioning knob.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2022/056681, which was filed Mar. 15, 2022, which application claimed priority from U.S. Provisional Application No. 63/163,164, filed Mar. 19, 2021, from European Patent Application Number 21173211.0, filed May 11, 2021, from U.S. Provisional Application No. 63/312,916, filed Feb. 23, 2022, and from U.S. Provisional Application No. 63/312,936, filed Feb. 23, 2022.

### FIELD OF THE INVENTION

A field of the invention includes catheter device for implanting a medical implant, such as a prosthetic heart valve or a medical occluder.

### BACKGROUND

A catheter device typically includes a capsule for receiving the implant when the latter is in a collapsed state; e.g., a crimped prosthetic heart valve such as an prosthetic aortic heart valve. The capsule covers the implant (e.g. a prosthetic aortic heart valve) that is positioned on a support element connected to an inner sheath of the catheter device while the capsule is connected to an outer sheath (the inner and outer sheaths may also be denoted as inner and outer shafts of the catheter).

Retraction of the outer sheath with respect to the inner sheath allows displacing the capsule with respect to the inner sheath and the support element so as to deploy and release said implant. With the catheter device of the present invention, an implant like a prosthetic aortic heart valve may be, e.g., only partially released and can be retracted into the capsule for the purpose of re-positioning the implant so that it can be deployed at a proper implantation site in a next attempt. Reinserting the implant into the capsule is commonly termed “resheathing” and is for example achievable by providing a connection of the implant to a connector connected to the inner sheath of the catheter device.

However, such a resheathing procedure is only possible up to a certain point of no return. Once this point has been passed upon deploying the prosthesis, the latter will be completely deployed and released from the catheter device and can no longer be retrieved by the catheter device.

Catheter devices of the afore-mentioned kind, particularly for implantation of a self-expanding transcatheter aortic valve replacement (TAVR) prosthesis (or TAVI prosthesis), are challenging in multiple aspects. Typical drawbacks encountered are a lack of ease of use, inaccurate positioning/orientation of the capsule at a target location, and, e.g., a lack of control regarding release of the implant from the inner sheath.

US 2019/0008640 A1 discloses a catheter device to provide improved steerability for delivering a prosthesis to a body location, for example, wherein the steerable delivery system can contain a steerable rail configured for multi-plane bending to direct a distal end of the delivery system. Furthermore, the handle of the device can include a communicative flush port, particularly a single flush port, for flushing out different lumens of the delivery system.

### SUMMARY OF THE INVENTION

A preferred catheter device of the invention can be a TAVR catheter device for a self-expanding aortic valve prosthesis, that allows implanting an implant, particularly a prosthetic heart valve, in particular a self-expanding aortic valve prosthesis, with the ability to control the axial position and angular orientation as well as its spatial orientation and the release and particularly the resheathing of the implant in an easy and safe manner.

A preferred catheter device (for implanting a medical implant) includes:
an outer sheath extending along a longitudinal axis of the catheter device and surrounding a lumen of the outer sheath;
an inner sheath extending along the longitudinal axis, wherein the inner sheath is arranged in the lumen of the outer sheath and connected to a support element for supporting the medical implant and/or a connector;
a capsule connected to a distal end of the outer sheath for covering the medical implant when the medical implant is arranged on the support element and or the connector;
a handle including: a grip portion for manually holding the handle, a rotatable deployment knob and a traveler, wherein the outer sheath is connected to the traveler and wherein the deployment knob is operatively connected to the traveler such that the traveler and thereby the outer sheath are moved along the longitudinal axis with respect to the inner sheath, and the handle optionally includes a rotatable axial positioning knob and a rotatable deflection knob; and
optionally a deflection sheath for adjusting the angular orientation of the capsule and/or the medical implant.

The deployment knob is preferably arranged adjacent the axial positioning knob, more preferably the deployment knob is arranged proximally to the axial positioning knob. The rotatable axial positioning knob is preferably situated between the rotatable deployment knob und the rotatable deflection knob. The grip portion is preferably situated proximally to the deployment knob.

A preferred catheter device for implanting a medical implant includes:
a deployable medical implant (for example a prosthetic heart valve such as an aortic prosthetic heart valve, but not limited thereto),
an outer sheath (also denoted as outer shaft) extending along a longitudinal axis of the catheter device and surrounding a lumen of the outer sheath,
an inner sheath (also denoted as inner shaft) extending along the longitudinal axis, wherein the inner sheath is arranged in the lumen of the outer sheath and connected to a support element at a distal portion of the inner sheath, wherein the support element is configured to support the medical implant,
a capsule connected to a distal portion of the outer sheath for covering the medical implant when the medical implant is arranged on the support element,
a deflection sheath (also denoted as deflection shaft) extending along the longitudinal axis and surrounding a lumen of the deflection sheath, wherein the deflection sheath is arranged in the lumen of the outer sheath and the inner sheath is arranged in the lumen of the deflection sheath (i.e. the deflection sheath is arranged between the inner and the outer sheath),
a handle including a stationary grip portion for manually holding the handle, a rotatable deployment knob, a rotatable axial positioning knob, and a rotatable deflection knob, wherein the deflection sheath is fixed to the grip portion and the deflection knob is operatively connected to a distal end section of the deflection sheath so that the deflection sheath and thereby the inner and the outer sheaths are deflected to adjust an angular orientation of the medical implant when the deflection knob is rotated in a first rotation direction of the deflection knob, and wherein the handle further includes a handle core, wherein the inner and the outer sheath are connected to the handle core and the axial positioning knob is operatively connected to the handle core such that the inner and the outer sheaths are simultaneously moved with the handle core with respect to the grip portion and the deflection sheath along the longitudinal axis when the axial positioning knob is rotated, and wherein the handle further includes a traveler, wherein the outer sheath is connected to the traveler, and wherein the deployment knob is operatively connected to the traveler such that the traveler and thereby the outer sheath are moved along the longitudinal axis with respect to the inner sheath, so as to deploy the medical implant when the deployment knob is rotated in a first rotation direction of the deployment knob.

### BRIEF DESCRIPTION OF THE DRAWINGS

In the following, features of the present invention are described in detail with reference to the Figures which show embodiments of the catheter device according to the present invention, wherein

FIG. 1 shows a perspective view of a handle of a catheter device for implanting a medical implant (preferably a prosthetic heart valve such as a prosthetic aortic heart valve) according to the present invention, wherein the handle includes a grip portion for manually holding the handle, a deployment knob for deploying the medical implant by moving the outer sheath with respect to the grip portion, an axial positioning knob for simultaneously moving the outer sheath and an inner sheath to which the implant is connected with respect to the grip portion of the handle in the axial direction of the catheter device (i.e. with respect to a longitudinal axis of the catheter device), and a deflection knob for adjusting an angular orientation of the medical implant;

FIG. 2A shows a catheter tip, a support element for the medical implant, and a connector, particularly a prosthesis connector, for connecting the medical implant to the inner shaft of the catheter device;

FIG. 2B further shows the capsule, outer sheath, and deflection sheath of a catheter device according to the present invention;

FIG. 2C shows a cut pattern of the wall of the inner sheath according to an embodiment of the present invention;

FIG. 2D shows a cut pattern of the wall of the outer sheath according to an embodiment of the present invention;

FIG. 2E shows a connection between an outer sheath and a capsule according to an embodiment of the present invention;

FIG. 2F shows an embodiment of a stent of a prosthesis that can be implanted using a catheter device according to the present invention;

FIG. 3 shows a schematic cross sectional view of a portion of an embodiment of a catheter device according to the present, wherein the medical implant is protected by a capsule connected to the outer sheath of the catheter device, and wherein the medical implant is connected to a connector at a distal end of the inner sheath; also shown is a stabilizing sheath (a so-called “stabilizer”) that is connected to the grip portion of the handle and a deflection sheath for adjusting the angular orientation of the capsule/medical implant;

FIG. 4 shows a schematical cross sectional view of the catheter device in the region of the capsule perpendicular to a longitudinal axis of the catheter device;

FIG. 5 shows a schematical cross sectional view of the catheter device in the region of the stabilizing sheath perpendicular to the longitudinal axis of the catheter device;

FIG. 6A shows a cross-sectional view of the handle of the catheter device to illustrate operation of the deflection knob of the handle;

FIG. 6B shows the capsule that connects to the handle of FIG. 6A;

FIG. 7A shows a cross sectional view of the handle of the catheter device to illustrate operation of the axial positioning knob;

FIG. 7B shows the capsule in two different axial positions that connects to the handle of FIG. 7A;

FIG. 8A shows a cross sectional view of the handle of the catheter device to illustrate operation of the deployment knob;

FIG. 8B shows the capsule that connects to the handle, wherein the capsule has been pulled back to deploy the implant;

FIG. 9 shows a cross-sectional view of the deployment knob and a traveler moveable by the deployment knob, wherein the outer sheath and therewith the capsule are connected to the traveler so that the implants can be deployed or re-sheathed by a corresponding rotation of the deployment knob;

FIGS. 10A-10B show perspective views of the traveler shown in FIG. 9;

FIG. 10C shows a further embodiment of a traveler of the catheter device according to the present invention;

FIGS. 11A-11D show a first stop and an actuation element arranged on the deployment knob for operating the first stop by which movement of the traveler in the proximal direction can be limited to ensure that the implant can be re-sheathed if during the procedure if necessary;

FIG. 12 shows different possible locations for a scale marking of an axial fine positioning indicator of the handle of a catheter device according to the present invention;

FIG. 13 shows an embodiment of a deflection indicator of the handle of a catheter device according to the present invention;

FIGS. 14A-14C show a deflection indicator for different deflection states of the deflection sheath of the catheter device; and

FIG. 15 shows an embodiment of a deployment indicator of the handle of a catheter device according to the present invention.

FIG. 16A shows a catheter tip, a support element for the medical implant, and a connector, particularly a prosthesis connector, for connecting the medical implant to the inner shaft of the catheter device;

FIG. 16B shows the catheter tip shown in FIG. 16A and a capsule for covering an implant arranged on the support element and connected to the prosthesis connector;

FIG. 17 shows a schematic cross sectional view of a portion of an embodiment of a catheter device according to the present invention, wherein the medical implant is covered by the capsule and connected to a connector at a distal end of the inner sheath; also shown is a stabilizing sheath that is connected to the grip portion of the handle (stabilizer) and a deflection sheath for adjusting the angular orientation/position of the capsule/medical implant;

FIG. 18A shows a cross sectional view of the handle of the catheter device to illustrate operation of the deployment knob;

FIG. 18B shows the capsule that connects to the handle, wherein the capsule has been pulled back to deploy the implant;

FIG. 19 shows a cross-sectional view of the deployment knob and a traveler moveable by the deployment knob, wherein the outer sheath and therewith the capsule are connected to the traveler so that the implant can be deployed or re-sheathed by a corresponding rotation of the deployment knob;

FIG. 20 shows a side view of an embodiment of a traveler according to the present invention;

FIG. 21 shows a helical groove of a deployment knob of an embodiment of a catheter device according to the present invention, wherein the helical groove includes a section having a reduced pitch for haptically indicating to the user that a complete deployment and release of the implant is imminent (point of no return); and

FIG. 22A-D show a mechanism of a further embodiment of a catheter device according to the present invention for indicating an imminent complete deployment and release of the implant to the user.

FIG. 23 shows a cross-sectional view of a distal section of an embodiment of a catheter device according to the present invention;

FIG. 24 shows a self-expandable scaffold, valve and skirt of a medical implant (e.g. TAVI heart valve prosthesis) that can be implanted with the catheter device shown in FIG. 23;

FIG. 25 shows a schematical cross-sectional view of the sheaths of the catheter device of FIG. 23;

FIG. 26 shows a schematical illustration of a flushing network to remove air from the second, third and fourth lumen, wherein the deflection sheath hub and the stabilizing sheath hub are stationary with respect to the second flushing port, and wherein the outer sheath hub is slidable relative to the second flushing port due to a telescopic connection; particularly, a check valve can be used to prevent fluid back flow from the outer sheath hub and the deflection sheath hub to the stabilizing sheath hub;

FIG. 27 shows the exit points of the lumens along the longitudinal axis of the catheter device and therewith the height of the liquid medium levels in the individual sheath;

FIG. 28A-B show a comparison of the hub positions during axial fine positioning, wherein the top image shows the most proximal, the bottom one shows the most distal position of the outer and inner sheaths; only the inner sheath and the outer sheath are moving;

FIG. 29A-B shows the movements of the hubs during deployment of the medical implant, wherein the top image shows the configuration with the capsule being closed, and wherein the bottom image shows the configuration with the capsule being open.

FIG. 30 shows the flow of the liquid medium (e.g. saline) through the flush lumen assembly, wherein the inner sheath (first lumen) is flushed via the first flushing port, and wherein all other lumens are flushed via the second flushing port;

FIG. 31 shows a cross-sectional view of the deflection sheath hub (left) and a detail of the telescopic connection (right);

FIG. 32 shows a cross-sectional view of the outer sheath hub; and

FIG. 33 shows an embodiment of a handle of the catheter device configured for moving the outer sheath hub and therewith the outer sheath and capsule, either together with the inner sheath for axial fine positioning of the capsule or for moving the outer sheath and capsule alone with respect to the inner sheath for deploying the medical implant.

### DETAILED DESCRIPTION OF THE PREFERRED EMBODIMENTS

A medical implant to be deployed by a catheter of the invention may be a heart valve prothesis, an aortic valve, a mitral valve, a pulmonal valve, a tricuspid valve, a (aortic) graft or a medical occluder like a left atrial appendage (LAA) occluder, an atrial or ventricular septal defect occluder or a patent foramen ovale occluder. Preferably, the catheter device for implanting a medical implant is a TAVI/TAVR delivery catheter system.

In the framework of the present invention, the notion “distal” refers to a portion or components of the catheter device that is remote from the handle or from the physician that operates the catheter device while the notion “proximal” refers to those portions or components that are closer to the handle or closer to the physician.

Preferably, the inner sheath, outer sheath, and the deflection sheath are flexible so that they are bendable in order to, e.g., conform to a curved course of a blood vessel. Accordingly, the longitudinal axis along which the catheter device extends from the handle to a distal catheter tip can include a corresponding curvature. Outside the human body, the sheaths and handle can be arranged along a straight line so that the longitudinal axis extends linearly.

According to an embodiment of the catheter device pursuant to the present invention, the grip portion is configured to be manually held by a user, so as to move the entire catheter device inside the body of the patient in order to position the medical implant (for example a prosthetic heart valve such as a prosthetic aortic heart valve) at the implantation site. Furthermore, according to an embodiment, the catheter device includes a stabilizing sheath (also denoted as stabilizing shaft or “stabilizer”) connected to the grip portion, wherein the stabilizing sheath surrounds a section of the outer sheath and is configured to reduce a friction between, e.g., an introducer and/or an anatomy of a patient, and the catheter device during the procedure; e.g. a TAVR intervention.

Particularly, according to an embodiment, the deflection sheath is connected to the grip portion, such that the inner and the outer sheaths slide along the deflection sheath when the axial positioning knob is rotated so that the angular orientation of the medical implant is maintained when the position of the inner and the outer sheaths along the longitudinal axis is adjusted by rotating the axial positioning knob.

Furthermore, according to an embodiment, the deflection knob is operatively connected to the distal end section of the deflection sheath via an elongated pulling member (for example a pull wire) and via an anchor of the handle, wherein the pulling member connects the distal end section of the deflection sheath to the anchor that is configured to be moved with respect to the grip portion along the longitudinal axis in a proximal direction when the deflection knob is rotated in the first rotation direction of the deflection knob such that the pulling member is tensioned and the deflection sheath is deflected to adjust an angular orientation of the medical implant.

Furthermore, the anchor is preferably configured to be moved with respect to the grip portion along the longitudinal axis in a distal direction when the deflection knob is rotated in an opposite second rotation direction of the deflection knob such that the pulling member is loosened and a deflection of the deflection sheath is reduced to adjust the angular orientation of the medical implant.

Furthermore, according to an embodiment of the present invention, the deflection knob includes a helical groove formed in a circumferential inside of the deflection knob, wherein the anchor includes a protrusion protruding from an outside of the anchor, wherein the protrusion engages with the helical groove such that the anchor is moved in the proximal direction when the deflection knob is rotated in the first rotation direction of the deflection knob and such that the anchor is moved in the distal direction when the deflection knob is rotated in the second rotation direction of the deflection knob. Particularly, the protrusion of the anchor is a helical protrusion including the same pitch as the helical groove of the deflection knob. Particularly, the helical groove can be an inner thread while the helical protrusion can be a corresponding outer thread of the anchor.

Further, according to an embodiment of the catheter device, the deflection knob is rotatably supported on a proximal end section of a support portion of the handle, wherein the support portion of the handle is connected via at least one elongated connecting member to the grip portion of the handle, wherein the at least one elongated connecting member extends along the longitudinal axis of the catheter device.

Furthermore, according to an embodiment of the invention, the proximal end section of the support portion of the handle includes a recess into which a distal end section of the deflection knob is inserted, wherein the recess of the support portion forms a slide bearing for the distal end section of the deflection knob.

Particularly, according to an embodiment, the support portion of the handle includes a circumferential protrusion protruding from a circumferential inside of the recess of the support portion, wherein this protrusion of the support portion of the handle engages in a form fitting manner with a circumferential groove formed in the distal and section of the deflection knob so that the distal end section of the deflection knob is held in the recess of the support portion of the handle while allowing rotating the deflection knob in the two opposite rotation directions (first rotation direction and second rotation direction) of the deflection knob.

Further, according to an embodiment of the catheter device, the axial positioning knob includes a helical groove formed in a circumferential inside of the axial positioning knob, and wherein the handle core includes a protrusion protruding from an outside of the handle core, wherein the protrusion of the handle core engages with the helical groove of the axial positioning knob to operatively connect the axial positioning knob to the handle core such that the handle core and therewith the inner sheath and the outer sheath are simultaneously moved with respect to the grip portion along the longitudinal axis in the distal direction when the axial positioning knob is rotated in a first rotation direction of the axial positioning knob, and such that the handle core and therewith the inner sheath and the outer sheath are simultaneously moved with respect to the grip portion along the longitudinal axis in the proximal direction when the axial positioning knob is rotated in an opposite second rotation direction of the axial positioning knob.

Further, according to an embodiment of the catheter device, the axial positioning knob is rotatably supported on a distal end section of the support portion of the handle, wherein the distal end section of the support portion of the handle forms a slide bearing for the axial positioning knob. In an embodiment, the axial positioning knob includes a circumferential protrusion protruding from the inside of the axial positioning knob, wherein the protrusion of the axial positioning knob engages with a circumferential groove formed in the distal end section of the support portion on an outside of the support portion of the handle so that the axial positioning knob is held on the distal end section of the support portion of the handle while allowing rotating the axial positioning knob in the two opposite rotation directions (first and second rotation direction) of the axial positioning knob.

Furthermore, according to an embodiment of the catheter device, the deployment knob is rotatably supported on the handle core and includes a helical groove formed in a circumferential inside of the deployment knob, wherein the traveler includes at least a first protrusion protruding from an outside of the traveler, wherein the at least one first protrusion engages with the helical groove of the deployment knob such that the traveler and thereby the outer sheath are moved in a proximal direction along the longitudinal axis of the catheter device with respect to the inner sheath and the handle core when the deployment knob is rotated in the first rotation direction of the deployment knob so that the capsule is pulled away from the medical implant in the proximal direction to deploy the medical implant and such that the traveler and thereby the outer sheath is moved in a distal direction along the longitudinal axis with respect to the inner sheath when the deployment knob is rotated in an opposite second rotation direction of the deployment knob, so that the capsule is pushed back over the implant to re-sheath a partially deployed medical implant or to close the empty capsule after final release of the implant. Preferably, according to an embodiment, the helical groove of the deployment knob is an inner thread.

Thus, particularly, the outer sheath is connected to the handle core via the traveler and the deployment knob. Preferably, the inner sheath is connected to a proximal end section of the handle core. Thus, when the deployment knob is not operated, the outer sheath is stationary with respect to the handle core, too.

Further, according to an embodiment of the catheter device, the traveler includes a second protrusion protruding from an outside of the traveler, wherein the first and the second protrusion engage with the helical groove of the deployment knob such that the traveler and thereby the outer sheath is moved in the proximal direction along the longitudinal axis with respect to the inner sheath when the deployment knob is rotated in the first rotation direction of the deployment knob, so that the capsule is pulled away from the medical implant in the proximal direction and such that the traveler and thereby the outer sheath is moved in the distal direction along the longitudinal axis with respect to the inner sheath when the deployment knob is rotated in the second rotation direction of the deployment knob, so that the capsule is pushed back over the implant to re-sheath the partially deployed medical implant or to close the empty capsule after final release of the implant.

Particularly, in an embodiment, the traveler includes a body having a top surface that forms a portion of the outside of the traveler, and a bottom surface that forms a portion of the outside of the traveler, too, which bottom surface faces away from the top surface.

Further, according to an embodiment of the catheter device, the top and the bottom surface of the traveler are spaced apart by a distance with respect to a first body axis of the body of the traveler, which first body axis extends perpendicular to the longitudinal axis, wherein the body further includes a first lateral surface and a second lateral surface, wherein the first lateral surface faces away from the second lateral surface, and wherein the two lateral surfaces are spaced apart by a distance with respect to a second body axis that extends perpendicular to the longitudinal axis of the catheter device and perpendicular to the first body axis, wherein the distance between the top and bottom surface with respect to the first body axis is larger than the distance between the first and second lateral side with respect to the second body axis.

According to a further embodiment of the catheter device, the first protrusion of the traveler protrudes from the top side, wherein the first protrusion preferably forms a curved wing having a first end protruding past the first lateral surface with respect to the second body axis, and a second end protruding past the second lateral surface with respect to the second body axis. Preferably, the curved wing is a helically curved wing (for example forms a section of a helix).

According to a further embodiment, the second protrusion of the traveler protrudes from the bottom side, wherein the second protrusion forms a curved wing having a first end protruding past the first lateral surface with respect to the second body axis, and a second end protruding past the second lateral surface with respect to the second body axis.

Alternatively, according to an embodiment, instead of a wing-like first and second protrusion, the first and the second protrusion can each include a pin or can be formed as a pin. Preferably, according to a further embodiment, the first and the second protrusion can each include a bearing, wherein the bearing of the first protrusion is arranged on the pin of the first protrusion, and wherein the bearing of the second protrusion is arranged on the pin of the second protrusion. The respective bearing is configured to engage into the helical groove of the deployment knob which advantageously reduces the friction between the traveler and the helical groove of the deployment knob.

Furthermore, according to an embodiment, the body of the traveler includes a through-hole extending along the longitudinal axis through the body of the traveler from a front surface of the body of the traveler to a back surface of the body of the traveler. Furthermore, according to an embodiment, the outer sheath includes a proximal end section that is arranged in the through-hole of the body of the traveler and connected to the body of the traveler.

Further, for limiting a movement of the traveler in the proximal direction upon rotating the deployment knob in the first rotation direction of the deployment knob, the deployment knob includes at least a first stop according to an embodiment, wherein the at least one first stop is configured to be arranged in an advanced position in which the first stop blocks the at least one first protrusion of the traveler so that further movement of the traveler in the proximal direction is prevented and the implant is kept in a partially deployed state in which the implant is still connected to the inner shaft.

Furthermore, according to an embodiment, the first stop is configured to be arranged in a retracted position for allowing further rotation of the deployment knob in the first rotation direction and corresponding movement of the traveler and the outer sheath along the longitudinal axis in the proximal direction so that the implant assumes a fully deployed state in which the implant is released from the inner sheath.

According to an embodiment, the deployment knob may further include a second stop that is configured to be arranged in an advanced position in which the second stop blocks the second protrusion of the traveler so that further movement of the traveler in the proximal direction is prevented and the implant is kept in the partially deployed state in which the implant is still connected to the inner shaft, and wherein the second stop is configured to be arranged in a retracted position allowing said further rotation of the deployment knob in the first rotation direction and said corresponding movement of the traveler and the outer sheath along the longitudinal axis in the proximal direction when the at least one first stop also resides in its retracted position.

Particularly, the first and the second stop are configured to be arranged in the respective advanced position and retracted position independently from the other stop. Furthermore, the first and the second stop are preferably arranged on opposite sides of the deployment knob with respect to a circumferential direction of the deployment knob.

Preferably, in an embodiment, the first stop is operatively connected to a manually operable first actuating element arranged on the deployment knob, wherein the first actuating element is configured to be manually operated to bring the first actuating element from a first state in which the at least one first stop is arranged in its advanced position to a second state in which the at least one first stop is arranged in its retracted position. Particularly, the actuating element can be a pushable button.

Furthermore, according to an embodiment of the present invention, the second stop is operatively connected to a manually operable second actuating element arranged on the deployment knob, wherein the second actuating element is configured to be manually operated to bring the second actuating element from a first state in which the second stop is arranged in its advanced position to a second state in which the second stop is arranged in its retracted position. Particularly, the second actuating element can be a pushable button, too.

Particularly, the first and the second actuating element are configured to be operated independently from one another.

Particularly, the first and the second actuating element are arranged on opposite sides of the deployment knob with respect to a circumferential direction of the deployment knob.

Furthermore, in an embodiment, the first actuating element is preloaded by a first spring towards the first state, so that the at least one first stop is returned to the advanced state when the first actuating element is released (e.g. not operated, particularly not being pushed).

Furthermore, in an embodiment, the second actuating element is preloaded by a second spring towards the first state, so that the second stop is returned to the advanced state when the second actuating element is released (e.g. not operated, particularly not being pushed).

Particularly, according to an embodiment, the first actuating element can be designed as a pivotable first actuating element including a first end section to which the at least one first stop is connected, a middle section that is pivotably supported on the deployment knob, and a second end section that is configured to be pushed against the action of the first spring to move the at least one first stop from its advanced position to its retracted position. The first spring can be arranged between a wall section of the deployment knob and the second end section of the first actuating element. Particularly, according to an embodiment, the first actuating element is arranged in a first recess formed in the outside of the deployment knob. Particularly, in the first state of the first actuating element, the first stop extends through a first lateral opening of the deployment knob into the helical groove of the deployment knob.

Particularly, according to an embodiment, the second actuating element can be designed as a pivotable second actuating element including a first end section to which the second stop is connected, a middle section that is pivotably supported on the deployment knob, and a second end section that is configured to be pushed against the action of the second spring to move the second stop from its advanced position to its retracted position. The second spring can be arranged between a wall section of the deployment knob and the second end section of the second actuating element. Particularly, according to an embodiment, the second actuating element is arranged in a second recess formed in the outside of the deployment knob. Particularly, in the first state of the second actuating element, the second stop extends through a second lateral opening of the deployment knob into the helical groove of the deployment knob. Particularly, the first and the second recess on the outside of the deployment knob face away from each other.

Further, according to an embodiment, the handle core forms a slide bearing for the deployment knob, wherein the deployment knob surrounds the handle core. Further, according to an embodiment, the deployment knob includes a distal circumferential protrusion protruding from the inside of the deployment knob, which distal circumferential protrusion engages in a form fitting manner with a distal circumferential groove formed in the outside of the handle core. Further, according to an embodiment, the deployment knob includes a proximal circumferential protrusion protruding from the inside of the deployment knob, which proximal circumferential protrusion engages in a form fitting manner with a proximal circumferential groove formed in the outside of the handle core. Thus, the deployment knob is held in place on the handle core with respect to the longitudinal axis but is rotatable about the longitudinal axis on the outside of the handle core. According to a further embodiment, a distal end section of the deployment knob extends into an orifice of the grip portion and is guided therein with respect to the longitudinal axis.

According to a further embodiment of the catheter device, the catheter device includes a connector, particularly a prosthesis connector, connected to a distal end of the inner sheath, wherein preferably the support element for the medical implant is connected to the inner sheath, particularly via the connector. Further, according to an embodiment, the connector is configured to engage with at least one fastening element of the medical implant, so as to connect the medical implant to the inner sheath when the medical implant is arranged on the support element.

Particularly, according to an embodiment, the catheter device, can include a guidewire and a respective guidewire lumen for guiding the capsule towards an implantation site.

Thus, according to an embodiment, the inner sheath surrounds a guidewire lumen for receiving the guidewire. According to an embodiment, the guidewire lumen is formed by a tubing that is surrounded by the inner sheath, wherein this tubing is connected, e.g. glued, to the inner sheath.

Further, according to an embodiment, the support element for carrying the medical implant is connected to a catheter tip. Particularly, the support element is connected to the catheter tip via said tubing. Furthermore, the catheter tip can include an opening formed in a distal end of the catheter tip, so that the guidewire can exit the guidewire lumen via said opening.

According to an embodiment, for allowing partial deployment of the medical implant and optional re-sheathing of the medical implant, the capsule includes a section (for example a distal end section) that is configured to cover the fastening element engaged with the connector when the at least one first protrusion butts against the at least one first stop and/or when the second protrusion butts against the second stop, so that said section of the capsule prevents the at least one fastening element to disengage from the connector. This can be achieved by maintaining the position of the at least one fastening element with respect to the connector by said section of the capsule such that the at least one fastening element remains engaged with the connector.

Further, according to an embodiment, the capsule is configured to be moved completely away from the at least one fastening element, so that said section of the capsule no longer covers the at least one fastening element and the at least one fastening element is set free and automatically disengages from the connector, e.g. due to its self-expanding capability, so that the medical implant assumes the fully deployed state. Hence, said automatic disengagement can be generated by a self-expanding property of the medical implant, e.g. a self-expanding property of a stent of the implant, which stent can be formed out of a shape-memory alloy such as Nitinol. Particularly, the at least one fastening element moves away from the connector and disengages the connector due to said self-expanding property once the capsule (particularly said section of the capsule) no longer covers the at least one fastening element, and thus no longer holds it in a fixed position with respect to the connector.

Particularly, according to an embodiment, the medical implant includes a self-expandable stent. The stent includes a collapsed state that can be achieved by crimping the stent to reduce its outer diameter. For implanting the medical implant the stent is positioned on the support element and is completely covered by the capsule and connected to the connector via the at least one fastening element of the stent. When the capsule is moved away from the stent, the stent and therewith the medical implant starts to deploy partially but remains connected to the connector as long as the capsule covers the fastening element and prevents it from disengaging with the connector. In the collapsed state, the stent/medical implant includes a diameter perpendicular to the longitudinal axis of the catheter device that is smaller than a diameter of the stent/medical implant perpendicular to the longitudinal axis when the stent and therewith the medical implant is in the fully deployed state.

Particularly, the at least one fastening element is connected to the stent or forms an integral portion of the stent, wherein the connector includes at least one recess or protrusion associated with the at least one fastening element, and wherein the at least one fastening element is configured to engage with the associated recess (or with the associated protrusion) so as to releasably connect the implant to the connector.

According to a preferred embodiment, the medical implant is a prosthetic heart valve, particularly a prosthetic aortic heart valve that can include said self-expandable stent. Furthermore, the medical implant can include valve leaflets (for example three valve leaflets) that can be connected to the stent. The valve leaflets can be formed out of a biological tissue, particularly a pliant biological tissue such as porcine or bovine pericardium.

In view of the above, in an embodiment, the prosthetic heart valve is configured for replacing an aortic heart valve. Preferably, the catheter device is a TAVR/TAVI catheter device, i.e. a catheter device for transcatheter aortic valve implantation (TAVI) or transcatheter aortic valve replacement, respectively. Here, the catheter device can be configured to deliver the prosthetic heart valve via one of the following accesses: transfemoral (in the upper leg), transapical (through the wall of the heart), subclavian (beneath the collar bone), transcarotid (through an incision in the neck), transaortic, i.e., direct aortic (through a minimally invasive surgical incision into the aorta), and transcaval (from a temporary hole in the aorta near the belly button through a vein in the upper leg).

Particularly, for flushing the lumina of the inner sheath and the outer sheath the catheter device can include at least two flushing ports connected to the handle according to an embodiment of the catheter device, particularly two flushing ports.

Further, according to an embodiment of the catheter device the deflection knob, the axial positioning knob, and the deployment knob each include a surface structure, wherein each surface structure differs from the other surface structures, so that each knob of the group included of the deflection knob, the axial positioning knob, and the deployment knob can be haptically identified by a physician by manually contacting the knobs. This is beneficial since the physician can for example focus on an intraoperatively acquired image of the patient/implantation side upon operating the individual knobs of the handle.

According to yet a further embodiment of the catheter device, each knob of the group included of the deflection knob, the axial positioning knob, and the deployment knob includes at least a first first indicative symbol indicating an adjustment of a function of the catheter device associated with the first rotation direction of the knob and at least a second indicative symbol indicating an adjustment of the function associated with the second rotation direction.

Using indicators, particularly including indicative symbols, the use of the functions of the handle can be shown on a surface of the handle surface to the user. Particularly, the indicators allow giving feedback to the user concerning a current state of a function such as axial positioning of the inner and outer sheath, deflection of the inner and outer sheath, deployment of the medical implant, and how much of the respective function can be used.

According to an embodiment of the catheter device according to the present invention, the handle includes an axial fine positioning indicator that is configured to indicate an axial position of the inner sheath and the outer sheath along the longitudinal axis regarding the simultaneous movement of the inner sheath and the outer sheath that can be generated by rotating the axial positioning knob.

This simultaneous movement of the inner and the outer sheath that corresponds to a simultaneous movement of the capsule and the catheter tip along the longitudinal axis, is also denoted as axial fine positioning (see above). Axial fine positioning (AFP) enables to precisely control the height of the medical implant at the target site. Particularly, regarding this simultaneous axial movement, the user needs to know how much of the travel has been used and how much more is available.

According to an embodiment, the axial fine positioning indicator includes a scale marking arranged on an outside of the distal end section of the deployment knob and a proximal end of the grip portion of the handle, wherein the scale marking moves with respect to the proximal end of the grip portion so that a current amount of a simultaneous travel of the inner and the outer sheath along the longitudinal axis can be inferred from the position of the scale marking with respect to the proximal end of the grip portion.

According to a further embodiment, the handle includes a deflection indicator for indicating a deflection of the deflection sheath and therewith an angular orientation of the medical implant.

Particularly, the shape, particularly curvature, of the catheter device is steered with help of the deflection sheath and deflection knob. Particularly, by rotating the deflection knob, the pulling member (e.g. pull wire) can be tensioned so as to bend the deflection sheath which results in adjusting said angular orientation of the medical implant. Preferably, the user needs to know what the current angular orientation is like. For example, the user has to deactivate the deflection function before or during the withdrawal of the catheter device from the aortic arch.

The deflection can be linked to the displacement of the anchor to which the pulling member is connected. Particularly, the anchor is tensioning the pulling member by moving in the proximal direction when the deflection knob is rotated in its first rotation direction.

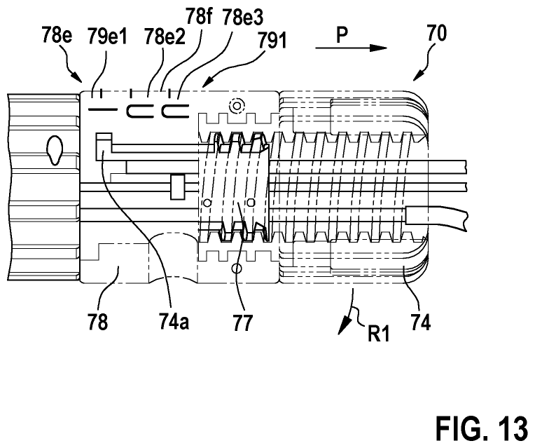

According to an embodiment as e.g. shown in FIG. 13, the deflection indicator includes an indicating member that is connected to the anchor (or alternatively arranged on the anchor) and configured to be moved along a scale marking included by the deflection indicator, which scale marking of the deflection indicator can be arranged on an outside of the support portion of the handle. Particularly, the support portion can be at least partially transparent so that the indicating member is visible from the outside below the scale marking when the anchor is moving due to rotating the deflection knob.

Furthermore, according to an embodiment, the handle of the catheter device includes a deployment indicator that is configured to indicate an axial position of the outer sheath with respect to the longitudinal axis regarding the movement of the outer sheath that can be generated by rotating the deployment knob.

The deployment function opens and closes the capsule that is covering the medical implant. In the correct position, the physician slowly opens the capsule until the implant is flared in the vessel but is not fully occluding the blood stream. If the position is still correct the physician continues the deployment to the point-of-no-return. Here, the prosthesis is already functional but can still be re-sheathed. If the position is good, the physician fully releases the implant. If the position is not satisfying, the physician can re-sheath. If additional travel is required to recapture the prosthesis, an overtravel can be used. All these states are realized with the movement of the outer shaft along the longitudinal axis of the catheter device. The current position of the capsule is visible under X-ray, but the deployment indicator on the handle significantly improves the handling of the catheter device.

The deployment state can be linked to the movement of the traveler, since the traveler takes along the outer sheath and therewith the capsule when the deployment knob is rotated.

Further, according to an embodiment, the deployment indicator includes an indicating member that is formed by the traveler (or connected to the traveler or arranged on the traveler) and configured to be moved along a scale marking included by the deployment indicator, which scale marking of the deployment indicator can be arranged on the deployment knob, particularly on the outside of the deployment knob.

Particularly, the deployment knob can be at least partially transparent, so that the indicating member is visible from the outside below the scale marking when the traveler is moving due to rotating the deployment knob.

A typical difficulty of the common catheter devices is to provide feedback to the user about the point of no return during the implant deployment. Particularly, it is desirable that this point during deployment of the prosthesis is clearly indicated to the user. At this point the user needs to decide if the implantation is satisfactory or if a resheathing or repositioning is required.

Another problem to be solved by the present invention is to provide a catheter device, particularly a TAVR (or TAVI) catheter device for a self-expanding aortic valve prosthesis or a catheter device for a medical occluder, that allows to clearly indicate the above-described point of no return to the user so that an unwanted complete deployment and release of the prosthesis can be avoided.

This problem is especially solved by a catheter device for implanting a medical implant including:
an outer sheath extending along a longitudinal axis of the catheter device and surrounding a lumen of the outer sheath,
an inner sheath extending along the longitudinal axis, wherein the inner sheath is arranged in the lumen of the outer sheath and connected to a support element for supporting the medical implant,
a capsule connected to a distal end of the outer sheath for covering the medical implant when the medical implant is arranged on the support element,
a handle including: a grip portion for manually holding the handle, a rotatable deployment knob, and a traveler, wherein the outer sheath is connected to the traveler, wherein the deployment knob includes a helical groove formed in an inside of the deployment knob, wherein the traveler includes at least a first protrusion protruding from an outside of the traveler, wherein the at least one first protrusion engages with the helical groove of the deployment knob such that the traveler and thereby the outer sheath are moved in a proximal direction along the longitudinal axis with respect to the inner sheath when the deployment knob is rotated in a first rotation direction, so that the capsule is pulled away from the medical implant in the proximal direction to deploy the medical implant, wherein for indicating an imminent complete deployment and release of the medical implant from the catheter device when the user rotates the deployment knob in the first rotation direction, the helical groove includes a section having a reduced (e.g. vanishing) pitch.

According to an alternative aspect of the present invention, for indicating an imminent complete deployment and release of the medical implant from the catheter device when the user rotates the deployment knob in the first rotation direction, the handle is configured to move the at least one protrusion out of the helical groove so that no further movement of the traveler along the longitudinal axis is allowed, particularly until reengagement of the at least one first protrusion into the helical groove is established by the user.

Particularly, said section of the helical groove is arranged between two adjacent sections wherein the pitch in said adjacent sections is larger than in said section of the helical groove. In other words, the pitch being reduced in said section means that a pitch of the helical groove before and after said section is larger than in said section. Preferably the pitch is zero in said section.

According to a preferred embodiment, the pitch is adapted such that a rotation of the deployment knob in the first rotation direction does not move the traveler and therewith the outer sheath in the proximal direction for a predefined fraction of a full rotation of the deployment knob in the first rotation direction when the at least one first protrusion engages with said section. According to a preferred embodiment, said pre-defined fraction is in the range from 0.1 to 0.75, wherein said fraction preferably amounts to 0.25, i.e. after having rotated the deployment knob by 10% to 75% (preferably 25%) of a full rotation (i.e. 360°), the traveler starts to move again in the proximal direction upon further rotation of the deployment knob in the first rotation direction and then causes full deployment and release of the implant without the possibility of re-sheathing of the implant.

According to a further embodiment, the at least one first protrusion engages with the helical groove of the deployment knob such that the traveler and thereby the outer sheath is moved in a distal direction along the longitudinal axis with respect to the inner sheath when the deployment knob is rotated in an opposite second rotation direction of the deployment knob, so that the capsule is moved back over the medical implant to resheath a partially deployed medical implant. Such a resheathing is possible in case the implant has not yet been completely deployed and released from the catheter device.

According to a further embodiment, the traveler includes a second protrusion opposite the at least one first protrusion, which second protrusion protrudes from the outside of the traveler, wherein the first and the second protrusion engage with the helical groove of the deployment knob such that the traveler and thereby the outer sheath are moved in a proximal direction along the longitudinal axis with respect to the inner sheath when the deployment knob is rotated in the first rotation direction of the deployment knob, so that the capsule is pulled away from the medical implant in the proximal direction to deploy the medical implant, wherein for indicating an imminent complete deployment and release of the medical implant from the catheter device when the user rotates the deployment knob in the first rotation direction, the helical groove includes a section having a pitch that is adapted such that a rotation of the deployment knob in the first rotation direction does not move the traveler and therewith the outer sheath in the proximal direction for a pre-defined fraction of a rotation of the deployment knob in the first rotation direction when the at least one first protrusion and the second protrusion engage with said section of the helical groove.

Particularly, according to an embodiment, the at least one first protrusion and the second protrusion protrude in opposite directions from an outside of the traveler, particularly perpendicular to the longitudinal axis of the catheter device.

According to a further embodiment, the traveler includes a body having a top surface and a bottom surface facing away from the top surface, wherein the at least one first protrusion of the traveler protrudes from the top surface, and wherein particularly the second protrusion of the traveler protrudes from the bottom surface of said body of the traveler.

Furthermore, in an embodiment, the at least one first protrusion is formed by a first pin or includes a first pin (particularly a cylindrical first pin) that is configured to engage into the helical groove of the deployment knob. Furthermore, according to an embodiment, the second protrusion is formed by a second pin or includes a second pin (particularly a cylindrical second pin) that is configured to engage into the helical groove of the deployment knob.

According to yet another embodiment, the at least one first protrusion includes a bearing that is configured to engage into the helical groove of the deployment knob to reduce friction between the traveler and the helical groove of the deployment knob. Likewise, the second protrusion can include a bearing that is configured to engage into the helical groove of the deployment knob to reduce friction between the traveler and the helical groove of the deployment knob.

According to a further preferred embodiment, for moving the at least one first protrusion out of the helical groove, the handle includes an elongated rod that extends along the longitudinal axis, wherein the traveler is configured to slide on said rod when the deflection knob is rotated in the first rotation direction or in the second rotation direction.

Furthermore, according to an embodiment of the present invention, the at least one first protrusion is connected to a first slider that is slidably arranged in a first opening of the body of the traveler and configured to slide on a surface of the rod.

Further, according to a preferred embodiment, the first slider is pretensioned against the surface of the rod, wherein a recess is formed in the surface of the rod such that when the deflection knob is rotated in the first rotation direction the first slider moves into the recess and the at least one first protrusion moves out of the helical groove when a complete deployment and release of the implant is imminent, which prevents further movement of the traveler and therewith of the outer sheath in the proximal direction when the deployment knob is rotated further in the first rotation direction.

According to a further preferred embodiment of the present invention, the rod is configured to be moved along the axial direction to push the first slider out of the recess of the rod and to thereby cause reengagement of the at least one first protrusion into the helical groove so that further rotation of the deployment knob in the first direction causes complete deployment and release of the implant from the catheter device.

Furthermore, according to an embodiment, the second protrusion of the traveler is connected to a second slider that is slidably arranged in a second opening of the body of the traveler and configured to slide on a surface of the rod, too.

Further, according to a preferred embodiment, the second slider is also pretensioned against the surface of the rod, such that when the deflection knob is rotated in the first rotation direction the at least one first slider and the second slider move into the recess (or each slider into one of two separate recesses of the rod) and the at least one first protrusion and the second protrusion move out of the helical groove when a complete deployment and release of the implant is imminent, which prevents further movement of the traveler and therewith of the outer sheath in the proximal direction when the deployment knob is rotated further in the first rotation direction.

According to a further preferred embodiment of the present invention, the rod is configured to be moved along the axial direction to push the first slider and the second slider out of the recess of the rod and to thereby cause reengagement of the at least one first protrusion and the second protrusion into the helical groove so that further rotation of the deployment knob in the first direction causes complete deployment and release of the implant from the catheter device.

The present invention offers the advantages of having a comparatively low complexity, a low maintenance, a short assembly time, as well as an easy handling of the components of the catheter device.

Particularly, the present invention is especially suitable for self-expandable delivery systems for TAVI or peripheral vascular intervention. The invention could generally be useful for any delivery system where an implant is released by a relative movement of an inner and an outer shaft and a limit should be indicated to the user, e.g. for self-expanding implants in bodily ducts such as blood vessels (stents, coils, occluders, particularly LAA, PFO, etc.), biliary ducts, urethra, esophagus, bronchia, etc., or also for implants such as leadless pacemakers, etc.

In order to avoid introduction of air bubbles into the vessel system of the patient upon implantation of the implant using the catheter device, the catheter device needs to be flushed with a suitable liquid medium, particularly a saline solution.

Another problem to be solved by the present invention is to provide a catheter device that enables an efficient flushing of all lumens with a suitable liquid medium like a saline solution. Particularly, it is desirable to have a small number of flushing ports as well as a proper sealing of the involved joints and connections. Particularly, it is further desirable to minimize the backflow so that after flushing all lumens remain flushed.

This problem is solved by a catheter device for implanting a medical implant including:
an inner sheath extending along the longitudinal axis and surrounding a first lumen (guidewire lumen) for receiving a guidewire,
a deflection sheath extending along the longitudinal axis and surrounding a second lumen, wherein the inner sheath extends through the second lumen,
an outer sheath extending along the longitudinal axis,
a capsule connected to a distal end of the outer sheath for covering the medical implant, wherein the outer sheath and the capsule together surround a third lumen, wherein the deflection sheath is arranged in the third lumen,
a stabilizing sheath extending along the longitudinal axis and surrounding a fourth lumen, wherein the outer sheath (as well as the inner sheath and the deflection sheath) extends through the fourth lumen, and
a handle configured to move the outer sheath and therewith the capsule with respect to the inner sheath, the deflection sheath, and the stabilizing sheath to release the implant, wherein the handle includes a first flushing port being in flow connection with the first lumen so as to allow flushing the first lumen with a liquid medium through the first flushing port, and wherein the handle includes a second flushing port being in flow connection with the fourth lumen, the second lumen, and the third lumen so as to allow flushing the fourth lumen, the second lumen, and the third lumen with a liquid medium through the second flushing port.

According to a preferred embodiment of the catheter device, the flow connection between the second flushing port and the fourth lumen, the second lumen, and the third lumen is configured such that a liquid medium injected into the second flushing port is split into a first and a second partial stream, the first partial stream flushing the fourth lumen, and wherein the second partial stream is split again into a third and a fourth partial stream, the third partial stream flushing the second lumen and the fourth partial stream flushing the third lumen.

Furthermore, according to an embodiment of the catheter device, the second flushing port is in flow connection with the third lumen via a telescopic connection including a first tube and a second tube so that a liquid medium (e.g. saline solution) can be passed through the second flushing port and the second tube and the first tube into the third lumen, wherein the first tube is configured to slide inside the second tube such that the outer sheath is movable with respect to the inner sheath, the deflection sheath and the stabilizing sheath while maintaining the flow connection between the second flushing port and the third lumen.

According to a further embodiment of the catheter device, the handle includes a grip portion for manually holding the handle.

Further, according to a preferred embodiment, the handle includes an outer sheath hub that is movable with respect to the grip portion of the handle to move the outer sheath with respect to the inner sheath (as well as with respect to the deflection sheath and the stabilizing sheath), wherein the outer sheath is connected with a proximal end to the outer sheath hub, and wherein the deflection sheath and the inner sheath extend through the outer sheath hub.

Furthermore, in an embodiment, the handle includes a deflection sheath hub that does not move with respect to the grip portion of the handle, wherein a proximal end of the deflection sheath is connected to the deflection sheath hub. Particularly, the outer sheath hub is movable with respect to the deflection sheath hub.

Furthermore, according to a preferred embodiment, a distal end of the first tube is connected to the outer sheath hub and wherein a distal end of the second tube is connected to the deflection sheath hub.

According to yet another preferred embodiment of the catheter device, the second flushing port is in flow connection with the deflection sheath hub via a flushing tube that includes a lateral opening that is in flow connection with the deflection sheath hub, wherein the deflection sheath hub provides a flow connection between said lateral opening and the second lumen of the deflection sheath and between said lateral opening and the first tube via the second tube. Furthermore, particularly, the outer sheath hub provides a flow connection between the first tube and the third lumen. Thus, liquid medium injected into the second flushing port is distributed via the deflection sheath hub into the second lumen of the deflection sheath and into the third lumen of the outer sheath.

Furthermore, according to an embodiment, the handle includes a stabilizing sheath hub that is stationary with respect to the grip portion of the handle, wherein the stabilizing sheath is connected with a proximal end to the stabilizing sheath hub, and wherein the outer sheath, the deflection sheath and the inner sheath extend through the stabilizing sheath hub.

Further, in an embodiment, said flushing tube mentioned above extends through the outer sheath hub to the stabilizing sheath hub and is connected with a distal end to the stabilizing sheath hub, wherein the stabilizing sheath hub provides a flow connection between the flushing tube and the fourth lumen of the stabilizing sheath so that liquid medium (e.g. a saline solution) injected into the second flushing port and passing through the flushing tube enters the fourth lumen of the stabilizing sheath.

Furthermore, particularly, the deflection sheath hub and the stabilizing sheath hub are stationary, i.e. do not move, with respect to one another and with respect to the grip portion of the handle.

Furthermore, according to an embodiment, the handle further includes a rotatable deployment knob, a rotatable axial positioning knob, and a rotatable deflection knob. Particularly, the deflection knob is operatively connected to a distal end section of the deflection sheath, so that the deflection sheath and thereby the inner and the outer sheaths are deflected to adjust an angular orientation of the capsule when the deflection knob is rotated in a first rotation direction of the deflection knob. Furthermore, preferably, the handle includes a handle core, wherein the inner sheath is connected to the handle core and wherein the outer sheath is connected to the handle core via the outer sheath hub. Further, particularly, the axial positioning knob is operatively connected to the handle core such that the inner and the outer sheath are simultaneously moved with the handle core with respect to the grip portion and the deflection sheath along the longitudinal axis when the axial positioning knob is rotated.

Furthermore, particularly, the deployment knob is operatively connected to the outer sheath hub such that the outer sheath hub and thereby the outer sheath are moved along the longitudinal axis with respect to the inner sheath for deploying the medical implant when the deployment knob is rotated in a first rotation direction of the deployment knob.

Particularly, the concept of a shiftable flush lumen assembly as described herein can be reduced to a simpler or expanded to a more complex system if the number and function of sheaths is changing.

Particularly, in case of a balloon-expandable device a lumen can be used to be filled with a fluid to expand the balloon of the device. Particularly, this lumen does not require additional flushing. The lumen(s) around the high-pressure lumen need to be flush and potentially moved axially.

Furthermore, in a catheter system with a steerable inner or outer sheath, the deflection sheath can be omitted and its function transferred to the inner our outer sheath. The complexity for flushing such a system is reduced since the number of lumens is reduced.

Furthermore, in a non-steerable catheter system that includes a relative motion of at least two sheaths can employ the telescopic connection described herein. This can be used for the flushing of lumens in any catheter system that is retrieving or advancing a sheath relative to another sheath.

The catheter device according this aspect of the invention simplifies the flushing process, increases safety and saves preparation time in an advantageous manner.

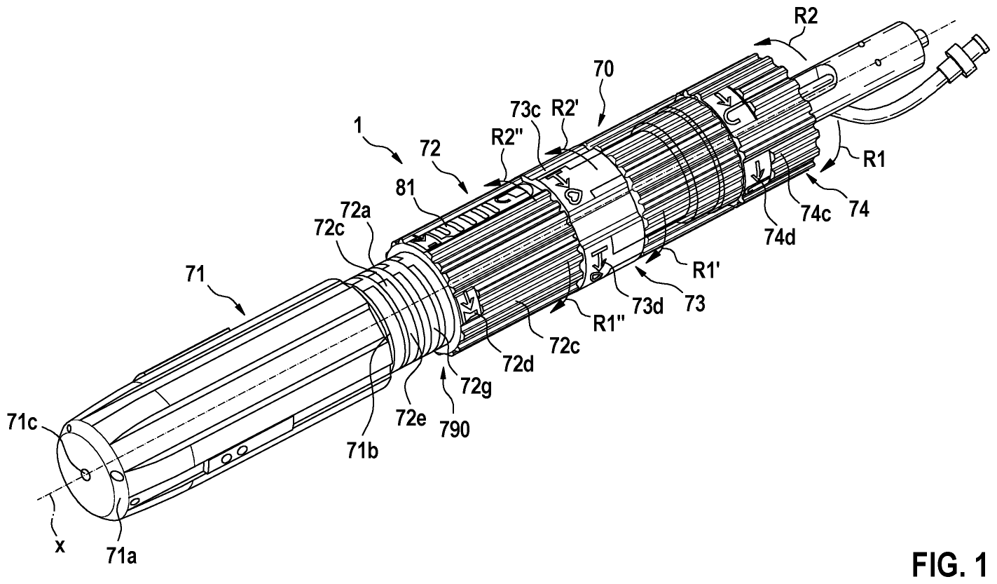

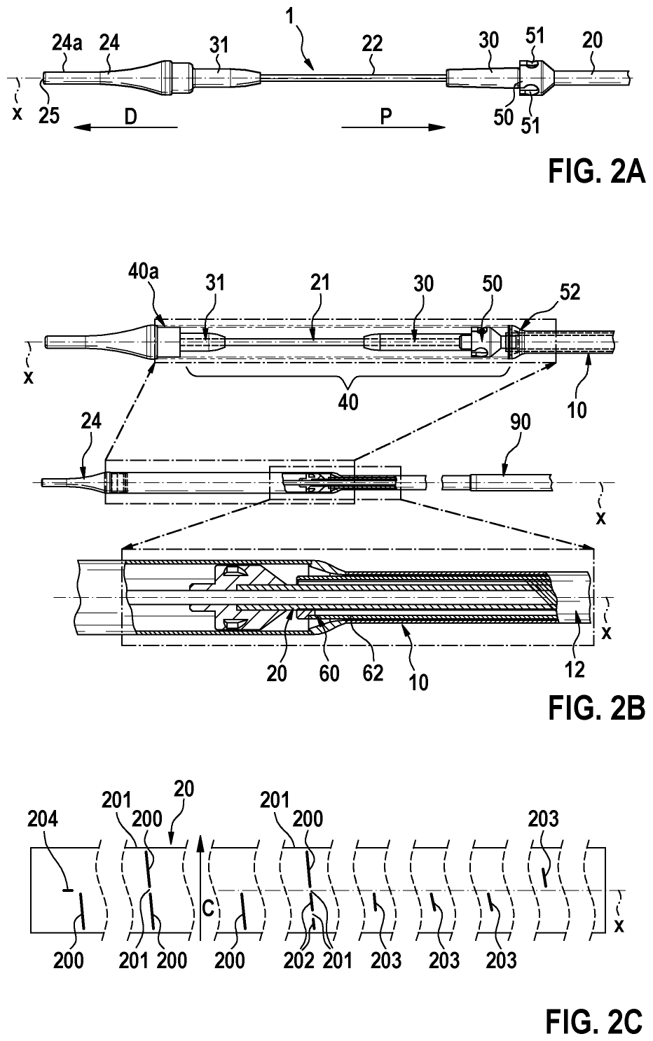

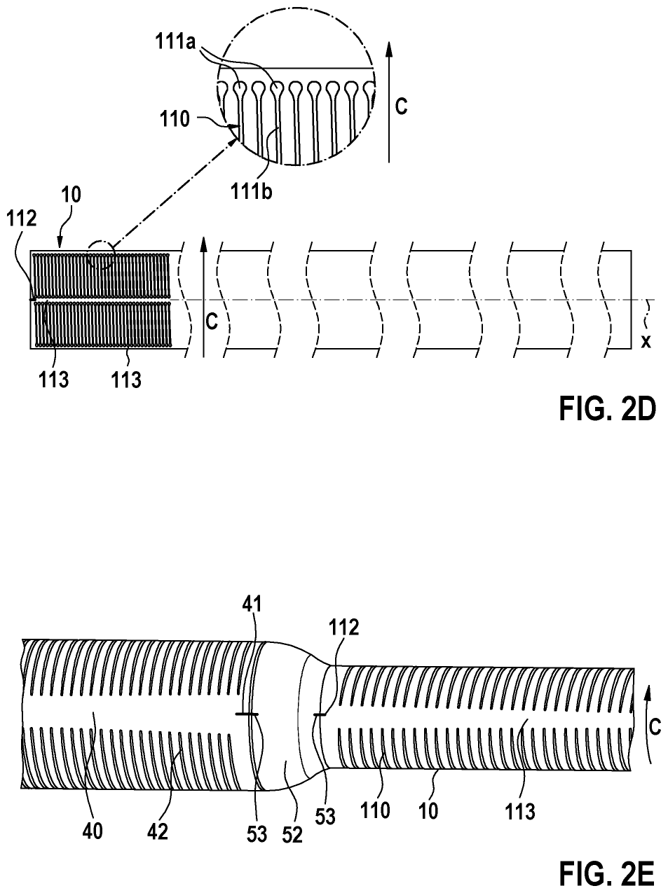

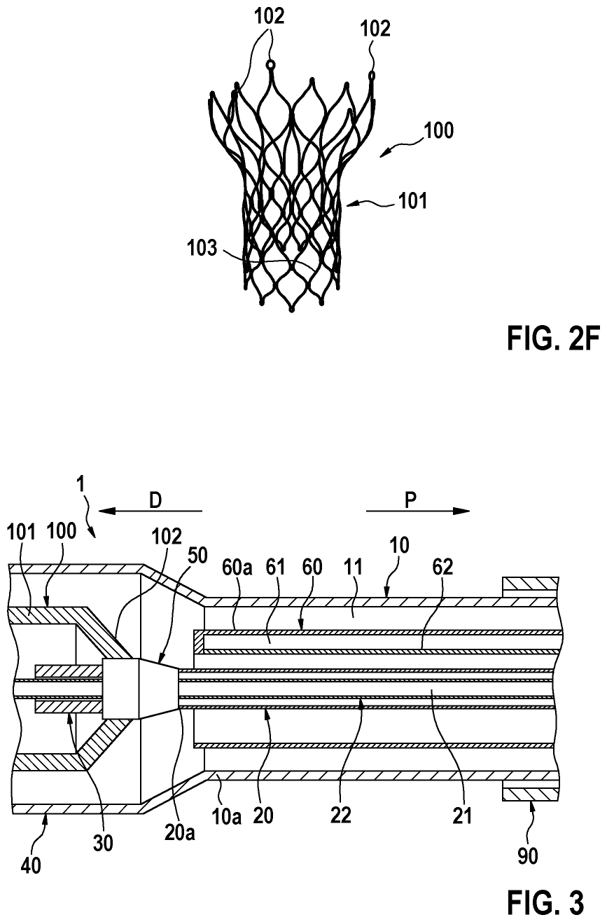

FIG. 1 shows an embodiment of a handle 70 of a catheter device 1 according to the present invention, the handle is particularly adapted to be used for a steerable triaxle catheter device which positions a medical implant 100, particularly a prosthetic heart valve prosthesis such as a prosthetic aortic heart valve that includes a self-expanding stent 101 with a tissue-based valve that can include three leaflets connected to the stent 101 that is shown in FIG. 2F for example in an expanded state, wherein the leaflets (not shown in FIG. 2F) can be made out of a biological tissue. The stent 101 preferably includes a plurality of interconnected struts 103, so that the stent 101 forms a circumferential scaffold including a plurality of lateral openings. The prosthetic heart valve 100 is particularly designed to replace a native aortic valve. Advantageously, the catheter device 1 according to the present invention facilitates prosthesis delivery as well as axial positioning and angular orienting. Furthermore, the handle enables partial deployment, full deployment and recapturing (resheathing) of the medical implant/prosthetic heart valve.

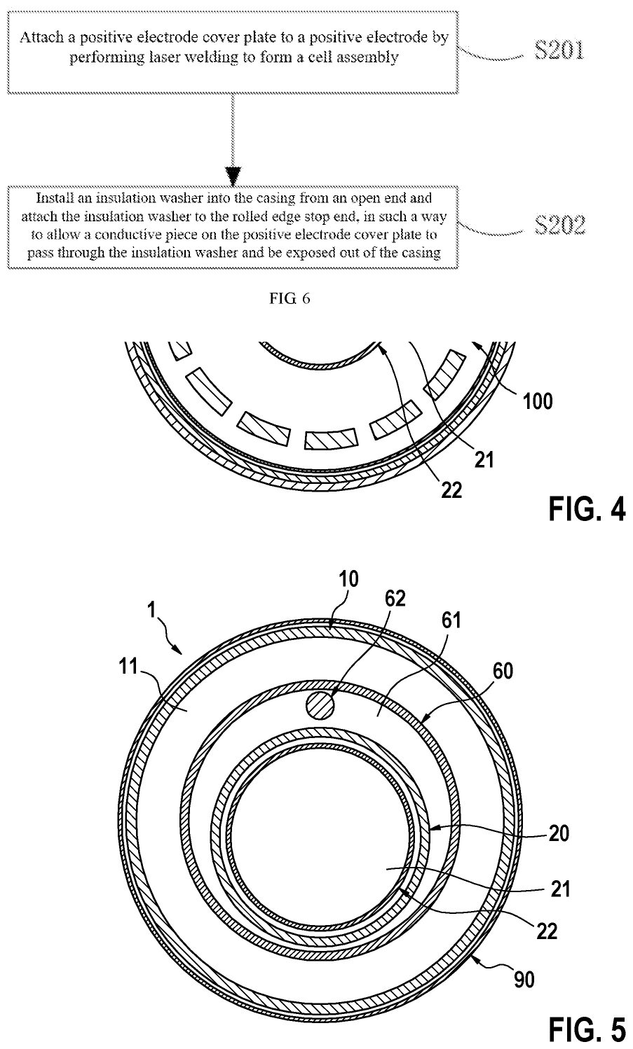

Particularly, the handle 70 is adapted to manipulate, particularly steer, four sheaths 20, 10, 60, and 90 that are shown in FIGS. 2B, 3, 4 and 5 for example, namely: an inner sheath 20 (also denoted as inner shaft 20) enclosing a guidewire lumen 21, a deflection sheath 60 (also denoted as deflection shaft 60) enclosing the inner sheath 20, an outer sheath 10 (also denoted as outer shaft 10) enclosing the inner sheath 20 and the deflection sheath 60, as well as a stabilizing sheath 90 (also denoted as stabilizing shaft or short “stabilizer” 90) enclosing a proximal section of the inner sheath 20, deflection sheath 60 and outer sheath 10. Particularly, each sheath 10, 20, 60 forms a tubular member.

For example, the outer sheath 10 can be formed out of a laser-cut stainless steel tube (so called hypotube) that can be covered with a polymer layer 12 (for example Pebax), cf. FIG. 2B. A friction reducer liner (e.g. PTFE or Polyimide) can be assembled to the inner surface of the outer sheath 10. Further, the deflection sheath 60 can be formed out of a laser-cut stainless steel tube (e.g. hypotube) and may include a friction reducer liner (e.g. PTFE or Polyimide) inside. The inner sheath 20 can also be formed out of a laser-cut stainless steel tube (e.g. hypotube), wherein the guidewire lumen 21 can be formed by a polymer tubing (e.g. PI) that can be glued into the inner sheath 20.

The three sheaths 10, 20, 60 can include different laser cut patterns. It is possible to use this configuration of sheaths 10, 20, 60 without polymer layers in between. However, using such layers in between the sheaths 10, 20, 60 allows preventing interlocking of the laser-cut tubes of the sheaths 10, 20, 60 to one another. These layers can be also useful as friction reducers during movement of the sheaths/tubes 10, 20, 60.

Furthermore, as indicated in FIG. 2B the capsule 40 can be connected to the outer sheath 10 via a capsule connector 52. Particularly, the distal end section 40a of the capsule 40 can be formed as a flaring crown 40a.

Furthermore, particularly, the inner sheath 20 can include a laser-cut stainless steel tube 20 including a cut pattern to form a double spine configuration at least in a distal portion of the tube 20 as shown in FIG. 2C, which shows the wall of the tube 20 being spread out in 2D. Particularly, the tube 20 includes a plurality of slots 200 in said distal portion, wherein for forming the double spine configuration, the slots 200 are arranged in pairs along a circumferential direction C of the wall of the inner sheath/tube 20 such that they are separated by two opposing wall sections 201 that form the double spine. The tube 20 can also include a middle portion having more than two slots 202 arranged along the circumferential direction C as well as a proximal portion including single slots 203 extending along the circumferential direction C. The pitch of the slots 200, 202, 203 can vary along the longitudinal axis x of the inner sheath/tube 20.

Furthermore, also the outer sheath 10 can include a laser-cut stainless steel tube 10 including a cut pattern to form a double spine configuration of the tube 10 as shown in FIG. 2D, which shows the wall of the outer sheath/tube 10 being spread out in 2D. Also here, the tube 10 includes a plurality of slots 110, wherein for forming the double spine configuration, the slots 110 are arranged in pairs along a circumferential direction C of the wall of the outer sheath/tube 10 such that they are separated by two opposing wall sections 113 that form the double spine. According to an embodiment, each slot 110 of the outer sheath/tube 10 includes two opposing rounded end sections 111a in which the respective slot includes a larger inner diameter compared to a middle section 111b of the respective slot 110.

Preferably, the outer sheath 10 and the capsule 40 each include an alignment marker 41, 112 (cf. FIGS. 2D and 2E), which can each be aligned with an associated alignment marker 53 of a capsule connector 52 that connects the outer sheath 10 to the capsule 40 as shown in FIGS. 2B and 2E. In this way, the cut patterns of the outer sheath 10 and of the capsule 40 can be precisely aligned with one another during assembly. Such line markers can also be provided on the inner sheath 20 and on the deflection sheath 60 for alignment purposes.

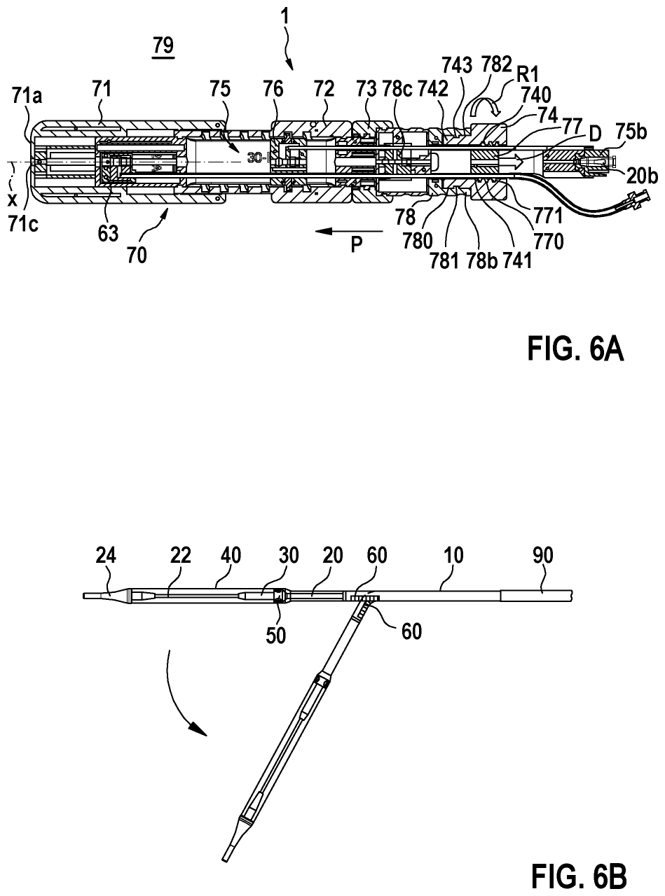

As indicated in FIGS. 2B, 3 and 5, the deflection sheath 60 is stationary and can be deflected with a pull wire 62 (for example a stainless steel wire) that can be connected to a distal end section 60a of the deflection sheath 60. The pull wire is arranged in a lumen of the deflection sheath 60. The deflection sheath 60 can include lateral openings so that the deflection sheath can be deflected by tensioning the pull wire 62. Particularly, in this way, the deflection sheath 60 can be deflected beyond 180°. In the letter case, the deflection sheath includes a u-shaped distal end section 60a (FIG. 6B shows a deflection of less than 90°).

Optionally, a friction reducer can be arranged between the inner sheath/tube 20 and the deflection sheath 60 (either connected to an outside of the inner sheath 20 or to an inside of the deflection sheath 60). The friction reducer can be formed out of PTFE or PI, and can be connected by gluing or reflow processing into or onto a sheath. In this way, it is also possible to apply a friction reducer only partially to a sheath. For example, a friction reducer in the form of a PI tube can be glued to an inside the deflection sheath 60.

Particularly, the catheter device 1 can further include a friction reducer, such as a PI tube, between the deflection sheath 60 and the outer sheath 10 that can e.g. either be connected to an outside of the deflection sheath 60 or to an inside of the outer sheath 10.

The inner and the outer sheath 20, 10 can be moved relative to the deflection sheath 60 (and relative to the stabilizing sheath 90). Additionally, the outer sheath 10 can be moved relative to the inner sheath 20. The stabilizing sheath 90 is stationary and fixed to a grip portion 71 of the handle 70. The stabilizing sheath 90 does only bridge the movements of the other sheaths 10, 20, 60 to the anatomy of the patient and/or an introducer, if necessary. Particularly, the deflection sheath 60 is shorter than the inner and the outer sheath 20, 10. Further, a capsule 40 can be connected to a distal end 10a of the outer sheath 10, wherein the capsule 40 can be larger in diameter (perpendicular to the longitudinal axis x along which the sheaths 10, 20, 60, 90 extend) than a proximal section of the outer sheath. The capsule 40 can include an outer jacket that can be formed out of a polymer (e.g. Pebax). Further, the capsule 40 can include a core that can be formed out of a laser-cut stainless steel tube (e.g. hypotube). An inner capsule liner may be formed out of PTFE, particularly with a polymer lamination layer (e.g. Pebax).

For delivery using the catheter device 1, the medical implant, here an aortic prosthetic heart valve 100, is placed on a support element 30 that is connected to a distal end section 20a of the inner sheath and covered by the capsule 40. The support element 30 can be connected to the inner sheath 20 via a connector 50 to which the heart valve prosthesis 100 is releasably connectable for delivery to an implantation site when it is arranged in the capsule 40. The guidewire lumen 21 can be formed by a tubing 22 that protrudes out of the inner sheath 20 at the distal end 20a of the inner sheath 20, extends through the connector 50 and support element 30, and connects to a catheter tip 24 to which the tubing 22 is connected. Furthermore, the catheter tip 24 can include an opening 25 formed in a distal end 24a of the catheter tip 24, so that the guidewire can exit the guidewire lumen 21 via said opening 25 (cf. FIGS. 2A and FIGS. 3 to 5).

The movements and functions of the catheter device 1 can be realized with the handle 70 of the catheter device 1. To this end, the handle 70 includes:
a stationary grip portion 71 configured for manually holding the handle 70 and to move the entire catheter device 1;
a rotatable deployment knob 72;
a rotatable axial (fine) positioning knob 73; and
a rotatable deflection knob 74.

Preferably, each of the knobs 72, 73, 74 is rotatable about the longitudinal axis x of the catheter device 1, along which axis x the handle extends.

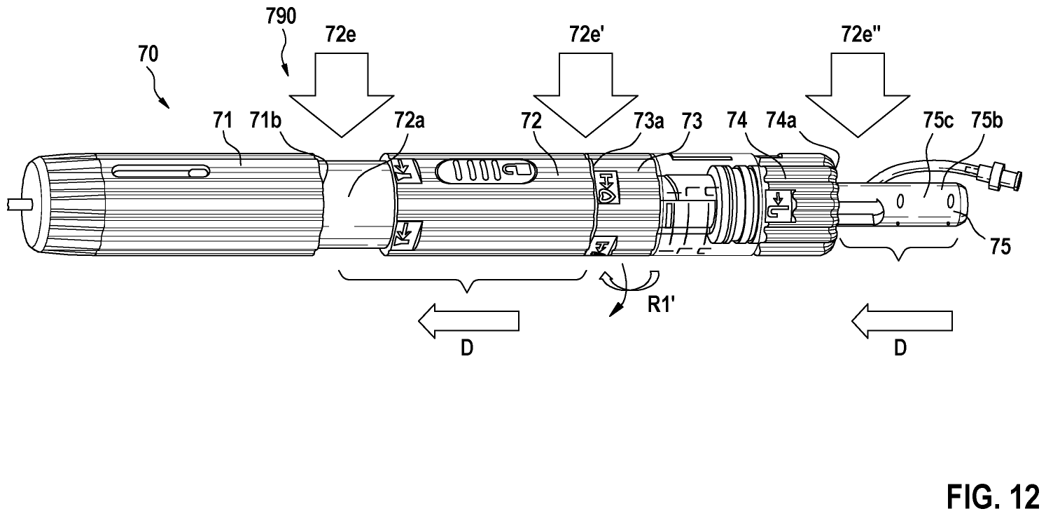

As can be seen in FIG. 6A, 7A, 8A and FIG. 12, the deployment knob 72 is arranged adjacent the axial positioning knob 73, preferably the deployment knob 72 is arranged proximally to the axial positioning knob 73. The rotatable axial positioning knob (73) is situated between the rotatable deployment knob (72) und the rotatable deflection knob (74). The grip portion is situated proximally to the deployment knob 72.

The deflection sheath 60 is connected to the grip portion 71, preferably via a hub 78c of the deflection sheath 60, which hub 78c is connected to a support portion 78 of the handle 70, wherein the support portion 78 is connected to the grip portion 71 (for example via an elongated member). The deflection sheath 60 is thus stationary with respect to the grip portion 71 and the support portion 78. Further, the stabilizing sheath 90 is connected to the grip portion, wherein the stabilizing sheath 90 is connected to a hub 63 of the stabilizing sheath 90, which hub 63 is connected to the grip portion 71 of the handle 70.

The grip portion includes an opening 71c at a distal end 71a of the grip portion 71 through which all sheaths 10, 20, 60, 90 extend into the handle 70 at the distal end 71a of the grip portion 71.

For deflecting the deflection sheath 60, the deflection knob 74 is operatively connected to a distal end section 60a of the deflection sheath 60, so that the deflection sheath 60 and thereby the inner and the outer sheath 20, 10 are deflected, i.e. bent, to adjust an angular orientation of the medical implant 100 when the deflection knob 74 is rotated in a first rotation direction R1 of the deflection knob 74 (cf. FIG. 1).

Further, for moving the inner and the outer sheath 20, 10 simultaneously the handle 70 includes a handle core 75 (cf. FIG. 6 to 9) that is movable with respect to the grip portion 71 along the longitudinal axis x, wherein the inner and the outer sheath 20, 10 are connected to the handle core 75 and the axial positioning knob 73 is operatively connected to the handle core 75 such that the inner and the outer sheath 20, 10 are simultaneously moved with the handle core 75 with respect to the grip portion 71 (and with respect to the deflection sheath 60 and stabilizing sheath 90) along the longitudinal axis x when the axial positioning knob 73 is rotated (for example in one of the rotation direction R1′, R2′; cf. FIG. 1).

Further, the handle 70 includes a traveler 76 (also denoted as outer sheath hub), wherein the outer sheath 10 is connected to the traveler 76, and wherein the deployment knob 72 is operatively connected to the traveler 76 such that the traveler 76 and thereby the outer sheath 10 are moved along the longitudinal axis x with respect to the inner sheath 20, and so to deploy the medical implant 100 when the deployment knob 72 is rotated in a first rotation direction R1″ of the deployment knob 72.

The catheter device 1 can be configured to be inserted into the patient via a transfemoral access and advanced to the target site. Once the catheter device 1 is roughly in place, the steerability feature controllable by the deflection knob 74 is used to orient the capsule 40 perpendicular and central to a patient's annulus plane. This is realized by the compression of the deflection sheath 60 with a pull wire 62 that is placed inside the lumen 61 of the deflection sheath 60. The deflection sheath 60 is stationary relative to the grip portion 71 of the handle 70. The pull wire 62 is fixed to an anchor 77 that is moved in proximal direction when the deflection knob 74 is rotated in the first rotation direction R1 of the deflection knob 74. The translation of rotation to axial displacement can be realized by a cam gear (for example a thread) as will be described further below. The movement of the anchor 77 along the longitudinal axis x in the proximal direction tensions the pull wire that is attached to the distal end 60a of the deflection sheath 60. The activation of the deflection does not move the inner and outer sheath 20, 10 along the longitudinal x but causes deflection of the inner and outer sheath 20, 10 as shown in FIG. 6B which shows the capsule 40 and implant 100 arranged therein as well as the sheaths 10, 20, 60, 90 that extend into the handle through the opening 71c of the grip portion 71.

In case no deflection (no tension of the pull wire 62) is applied, the inner and outer sheath 20, 10 extend along a straight line/longitudinal axis x. This configuration can be used for loading, preparation and tracking of the device 1 to the aortic arch.

By applying tension to the pull wire 62 by way of rotating the deflection knob in the first rotation direction R1, a 90° curve of the inner and outer sheath 20, 10 can be achieved. FIG. 6B shows a situation where such a 90° is about to be realized. Such a deflection of the inner and outer sheath 20, could assist the tracking around the aortic arch. The bending of the catheter device 1 reduces the contact and friction to the vessel wall. The risk of particle release or damage of the vessel wall is thus reduced.

Further tensioning of the pull wire 62 causes a larger deflection (e.g. more than 180°) of the deflection sheath 60 and thus of the inner and outer sheath 20, 10 that is also denoted as “candy cane”. This corresponds to a final adjustment of the angular position of the implant 100 at the implantation site. The goal is to position the prosthesis 100 perpendicular to and centered in a patient's annulus plane. Loosening the pull wire 62 by rotating the deflection knob 74 in the second rotation direction R2 reduces deflection of the inner and outer sheaths 20, 10.

In order to move the anchor 77, the deflection knob 74 includes a helical groove 740 (for example an inner thread) formed in a circumferential inside 741 of the deflection knob 74 as shown in FIG. 6A, wherein the anchor 77 includes a helical protrusion 770 (for example an outer thread) protruding from an outside 771 of the anchor 77. Both, the helical groove 740 and the protrusion 770 extend around the longitudinal axis x, wherein a rotation axis of the deflection knob 74 is aligned with the longitudinal axis x of the catheter device 1. The protrusion 770 engages with the helical groove 740 such that the anchor 77 is moved with respect to the grip portion 71 of the handle 70 in the proximal direction P when the deflection knob 74 is rotated in the first rotation direction R1 and such that the anchor 77 is moved in the distal direction D when the deflection knob 74 is rotated in the second rotation direction R2.

As shown in FIG. 6A, the deflection knob 74 can be rotatably supported on a proximal end section 78b of the support portion 78 of the handle 70. Particularly, the proximal end section 78b of the support portion 78 of the handle 70 includes a recess 780 into which a distal end section 742 of the deflection knob 74 is inserted, so that the deflection knob 74 can rotate in the recess 780. Particularly the recess 780 forms a slide bearing for the deflection knob 74. Particularly, the support portion 78 of the handle 70 includes a circumferential protrusion 781 protruding from a circumferential inside 782 of the recess 780, wherein this protrusion 781 engages in a form fitting manner with a circumferential groove 743 formed in the distal and section 742 of the deployment knob 74, so that the distal end section 742 of the deployment knob 74 is held in the recess 780 of the support portion 78 of the handle 70 while allowing rotating the deployment knob 74 in the two opposite rotation directions R1, R2 of the deployment knob 74.

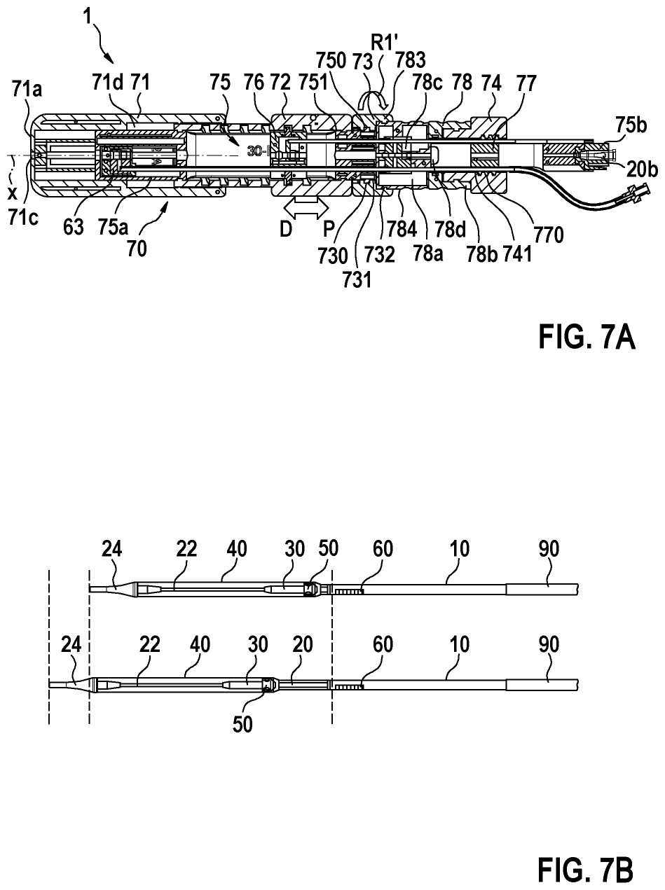

Further, the height of the implant 100 can be controlled by the axial positioning feature of the catheter device 1 that is controllable by the axial positioning knob 73. The catheter device 1 allows the axial advancement in the distal direction D as well as an opposite movement in the proximal direction P of the inner and outer sheaths 20, 10 relative to the grip portion 71 of the handle 70, and therefore also relative to the deflection and stabilizing sheaths 60, 90. Two different axial positions of the capsule generated by the axial positioning feature are shown in FIG. 7B.

The rotation of the axial positioning knob 73 moves the handle core 75 via a cam gear 730, 750. The handle core 75 is engaged with a fixation of the inner shaft 20 and a fixation of the outer shaft 10. Particularly, the inner sheath 20 is connected with a proximal end section 20b of the inner sheath 20 to a proximal end section 75b of the handle core 75. The outer sheath 10 is connected to handle core 75 via the traveler 76 which is operatively connected to the deployment knob 72 that is rotatably supported on the handle core 75.

Particularly, said cam gear includes a helical groove 730 formed in a circumferential inside 731 of the axial positioning knob 73 and a protrusion 750 protruding from an outside 751 of the handle core 75, wherein the protrusion 750 of the handle core 75 engages with the helical groove 730 of the axial positioning knob 73 to operatively connect the axial positioning knob 73 to the handle core 75 such that the handle core 75 and therewith the inner sheath 20 and the outer sheath 10 are simultaneously moved with respect to the grip portion 71 along the longitudinal axis x in the distal direction D when the axial positioning knob 73 is rotated in a first rotation direction R1′ of the axial positioning knob 73, and such that the handle core 75 and therewith the inner sheath 20 and the outer sheath 10 are simultaneously moved with respect to the grip portion 71 of the handle 70 along the longitudinal axis x in the proximal direction P when the axial positioning knob 73 is rotated in an opposite second rotation direction R2′ of the axial positioning knob 73.

As shown in FIG. 7, the axial positioning knob 73 is rotatably supported on a distal end section 78a of the support portion 78 of the handle 70, wherein the distal end section 78a of the support portion 78 of the handle 70 forms a slide bearing for the axial positioning knob 73. Further, the axial positioning knob 73 includes a circumferential protrusion 732 protruding from the inside 371 of the axial positioning knob 73, wherein the protrusion 732 of the axial positioning knob 73 engages in a form fitting manner with a circumferential groove 783 formed in the distal end section 78a of the support portion 78 on an outside 784 of the support portion 78 of the handle 70, so that the axial positioning knob 73 is held on the distal end section 78a of the support portion 78 of the handle 77 while allowing rotating the axial positioning knob 73 in the two opposite rotation directions R1′, R2′ of the axial positioning knob 73.

Preferably (cf., e.g., FIG. 7A), the grip portion 71 and the supporting portion 78 of the handle 70 each surround an orifice 71d, 78d, respectively, wherein the respective orifice 71d, 78d extends along the longitudinal axis x of the catheter device 1. The handle core 75 is slidably arranged with a distal end section 75a in the orifice 71d of the grip portion 71. Furthermore, the handle core 75 is slidably arranged with its proximal end section 75b in the orifice 78d of the support portion 78 of the handle 70, wherein a part of the proximal end section 75b of the handle core 75 protrudes out of the orifice 78d of the support portion 78 in the proximal direction P. This allows easy access to the proximal end section 20b of the inner sheath 20 that is connected to the proximal end section 75b of the handle core 75.

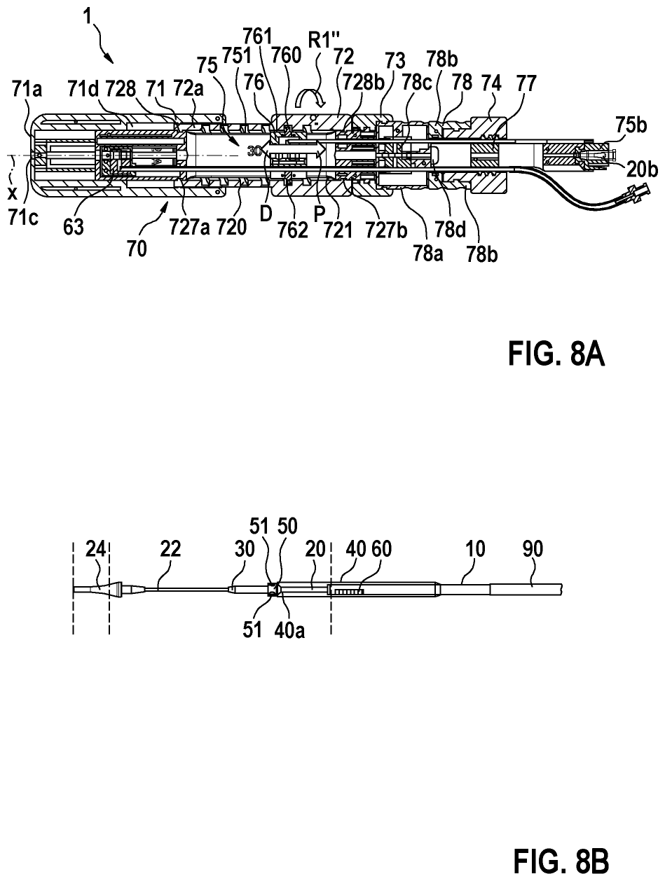

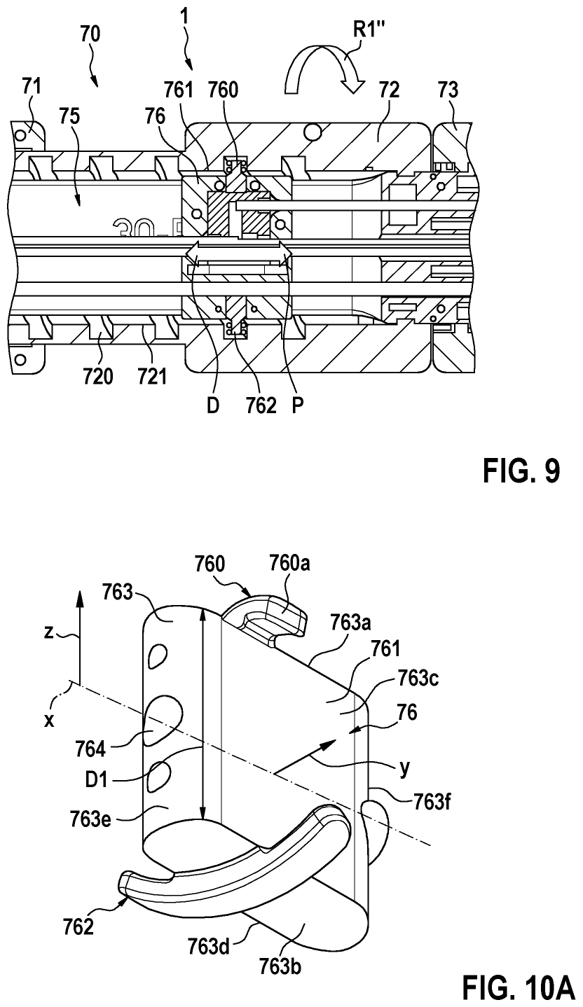

If the physician is satisfied with the positioning of the catheter device 1, deployment of the prosthesis 100 is started by the physician. In this regard, an axial movement of the outer sheath 10 (and therewith of the capsule 40 and implant 100) in the proximal direction P relative to all other sheaths 20, 60, 90 and the grip portion 71 of handle 70 releases the implant 100; e.g. a prosthetic aortic heart valve. This movement of the outer sheath 10 can be controlled by the deployment knob 72 (cf. FIGS. 8 and 9), wherein rotation of the deployment knob 72 moves the traveler 76 to which the outer sheath 10 is connected. The movement of the outer sheath 10 and therewith of the capsule 40 in the proximal direction P releases the implant 100.

For releasing the implant 100 based on the above-described movement of the outer sheath 10 and capsule 40, the connector 50 connected to a distal end 20a of the inner sheath 20 includes a recess 51, wherein the least one fastening element 102 of the medical implant 100 is engaged with the at least one recess 51 as long as the capsule 40 covers the at least one recess 51 of the connector 50 and the at least one fastening element 102 that engages with the at least one recess 51 when the prosthetic heart valve 100 is arranged on the support 30 (cf. FIGS. 2 and 3). The at least one fastening element 102 can be formed by a portion of the self-expandable stent 101 of the prosthetic heart valve 100. Particularly, as shown in FIG. 2F, the at least one fastening element 102 can be formed as an eyelet, that is connected to at least one strut 103 of the stent 101 at a proximal end of the stent 101. For example, the stent 101 can include three such fastening elements 102. In this case, the connector 50 includes three corresponding recesses 51.

Once the capsule 40 is completely removed from the implant 100 and does no longer cover the at least one fastening element 102 and the corresponding recess 51 of the connector 50 (cf. FIG. 8B) the at least one fastening element 102 disengages with the connector 50 due to the self-expanding property of the implant 100/stent 101, which releases the implant 100 at the implantation site.

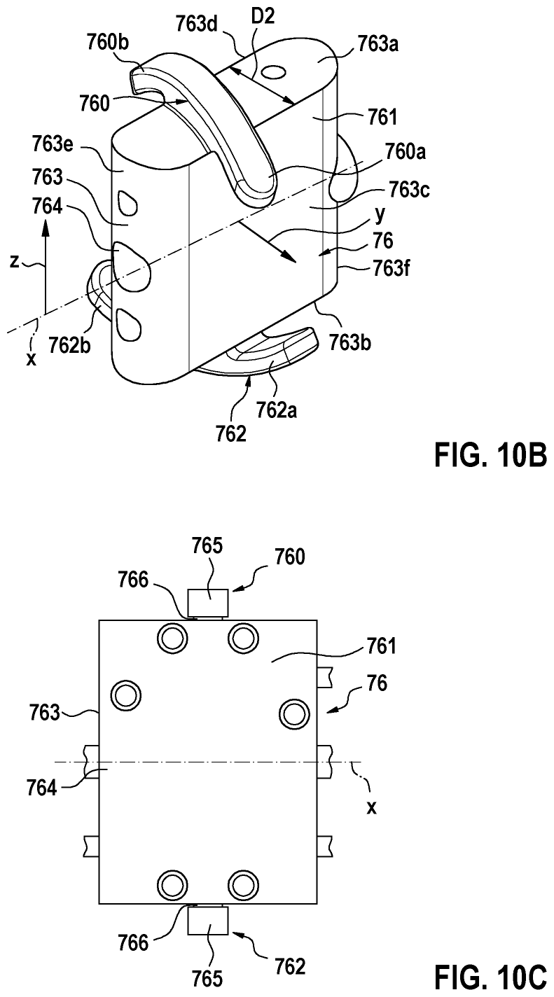

For operatively connecting the deployment knob 72 with the traveler 76, the deployment knob 72 is rotatably supported on the handle core 75 and includes a helical groove 720 formed in a circumferential inside 721 of the deployment knob 72 as shown in FIGS. 8 and 9, wherein the traveler 76 preferably includes a first protrusion and a second protrusion 760, 762 protruding from an outside 761 of the traveler 76 (cf. FIGS. 10A and 10B), respectively, wherein the protrusions 760, 762 engage with the helical groove 720 of the deployment knob 72 such that the traveler 76 and thereby the outer sheath 10 are moved in a proximal direction P along the longitudinal axis x with respect to the inner sheath 20 when the deployment knob 72 is rotated in the first rotation direction R1″ of the deployment knob 72, so that the capsule 40 is pulled away from the medical implant 100 in the proximal direction P to deploy the medical implant 100.

Particularly, the handle core 75 can form a slide bearing for the deployment knob 72, which surrounds the handle core 75 according to FIGS. 8 and 9. The deployment knob 72 can include a distal circumferential protrusion 727a protruding from the inside 721 of the deployment knob 72, which protrusion 727a engages in a form fitting manner with a distal circumferential groove 728 formed in the outside 751 of the handle core 75. Further, the deployment knob 72 can include a proximal circumferential protrusion 727b protruding from the inside 721 of the deployment knob 72, which protrusion 727b engages in a form fitting manner with a proximal circumferential groove 728b formed in the outside 751 of the handle core 75. The deployment knob 72 is thus fixed to the core 75 with respect to the longitudinal axis x but is allowed to rotate about the longitudinal axis x on the outside 751 of the handle core 75. A distal end section 72a of the deployment knob 72 extends into the orifice 71d of the grip portion 71 and can be guided therein—like the distal end section 75a of the handle core 75—along the longitudinal axis x.

Particularly, as shown in FIGS. 10A and 10B, the traveler 76 includes a body 763 having a top surface 763a that forms a portion of the outside 761 of the traveler 76 and a bottom surface 763b that forms a portion of the outside 761 of the traveler 76, too, wherein the bottom surface 763b faces away from the top surface 763a.

Further, the top and the bottom surfaces 763a, 763b, are spaced apart by a distance D1 with respect to a first body axis z of the body 763 that extends perpendicular to the longitudinal axis x of the catheter device 1, wherein the body 763 further includes a first lateral surface 763c and a second lateral surface 763d, wherein the first lateral surface 763c faces away from the second lateral surface 763d, and wherein the two lateral surfaces 763c, 763d are spaced apart by a distance D2 with respect to a second body axis y that extends perpendicular to the longitudinal axis x and perpendicular to the first body axis z, wherein the distance D1 between the top and bottom surface 763a, 763b is larger than the distance D2 between the first and second lateral surface 763c, 763d.

As further indicated in FIGS. 10A and 10B, the first protrusion 760 of the traveler 76 protrudes from the top surface 763a and preferably forms a helically curved wing 760 having a first end 760a protruding past the first lateral surface 763c with respect to the second body axis y, and a second end 760b protruding past the second lateral surface 763d with respect to the second body axis y, i.e. the extension of the wing 760 in the direction of the second body axis y is larger than the distance D2. The second protrusion 762 protrudes from the bottom surface 763b, wherein the second protrusion 762 forms a helically curved wing having a first end 762a protruding past the first lateral surface 763c with respect to the second body axis y, and a second end 762b protruding past the second lateral surface 763d with respect to the second body axis y. Also here, the extension of the wing 762 in the direction of the second body axis y is larger than the distance D2.

As shown in FIG. 10C, instead of the wing-like protrusions 760, 762 shown in FIGS. 10A and 10B, one can also use the traveler 76 as shown in FIG. 10C with alternative protrusions 760, 762. For example each protrusion 760, 762 can include a bearing 765 that can be supported on a pin 766 protruding from the body 763 of the traveler 76, wherein the respective bearing 765 is rotatable about a rotation axis that extends perpendicular to the longitudinal axis x for reducing friction when the projections 760, 762 engage into the helical groove 720 of the deployment knob 72 via the bearings 765. Alternatively, one may also use protrusions 760, 762 in the form of pins, i.e., without bearings thereon.

The respective bearing 760, 762 is configured to engage into the helical groove 720 of the deployment knob 72 which advantageously reduces the friction between the traveler 76 and the helical groove 720 of the deployment knob 72.

As further indicated in FIGS. 10A, 10B and 10C, the body 763 of the traveler 76 includes a through-hole 764 extending along the longitudinal axis x of the catheter device 1 through the body 764 of the traveler 76 from a rounded front surface 763e of the body 763 of the traveler 76 to a rounded back surface 763f of the body 763 of the traveler 76. The through-hole 764 serves for fixing the outer sheath 10 to the traveler 76. To this end, the outer sheath 10 includes a proximal end section that is arranged in the through-hole 764 and connected to the body 763 of the traveler 76. Particularly, the sheaths 20, 60 and tubing 22 inside the outer sheath 10 pass all the way through the through-hole 764.

By rotating the deployment knob 72, the user is deploying the prosthesis 100 up to a point-of-no-return at which the capsule 40 merely covers the at least one fastening element 102 with a distal end section 40a of the capsule 40 to prevent it from moving out of the associated recess 51 of the connector 50. The remaining part of the implant 100, particularly the stent 101, is partially deployed at this stage and the user can assess the performance of the partially deployed implant 100 and still has the option to recapture (re-sheath) the implant 100, i.e. move the capsule 40 back over the implant by rotating the deployment knob in the opposite second direction R2″. Once the user overcomes this point the implant 100 has to be fully deployed and cannot change its position anymore.

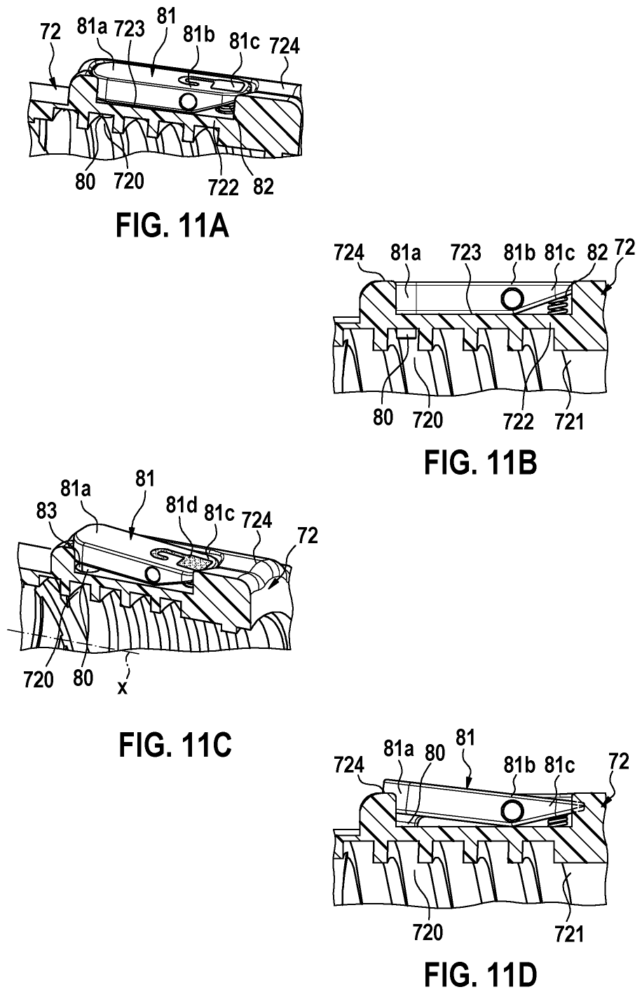

For safety reasons, this re-sheathing limit is indicated by a hard-stop including two individual and independently operable stops 80 that can preferably only be overcome if the user operates two independent actuating elements 81 (for example in the form of pushable buttons). These actuating elements 81 are preferably 180° offset with respect to the periphery of the deployment knob 72. One of such an actuating element 81 and an associated stop 80 is shown in FIG. 11A to 11D, the other actuating element 81 and stop 80 associated thereto are not shown, but are preferably arranged on the other side of the deployment knob 72, i.e. 180° offset with respect to the periphery of the deployment knob 72, particularly in a mirror symmetrical fashion with regard to the longitudinal axis x.

The traveler 76 that is fixedly connected to the outer sheath 10 is moving within the helical groove (e.g. inner thread) 720 of the deployment knob 72 (cf. FIG. 9). As described above, the traveler 76 has two wing-like protrusions 760, 762 that are reaching into the helical groove 720 of the deployment knob 72. The stops 80 are designed to block the movement of the traveler's protrusions 760, 762 once it reaches the re-sheathing limit. Each stop 80 blocks one of the protrusions 760, 762, so that the user has to operate both actuating elements 81 to remove the obstacle in the groove/thread 720 and allow the traveler 76 to move further proximal which releases the then fully deployed implant 100.

Particularly, as illustrated in FIGS. 11A to 11D, the first and the second stop 80 are each configured to be arranged in an advanced position, in which the respective stop 80 protrudes through an associated opening 83 of the deployment knob 72 into the groove 720 to block the associated protrusion/wing 760, 762 of the traveler 76 (cf. FIG. 9). When the respective traveler protrusion 760, 762 butts against the associated stop 80 the re-sheathing limit has been reached and merely the distal end section 40a of the capsule 40 still covers the at least one fastening element 102 and prevents it from being released from the connector 50 (see also above).

Each stop 80 is further configured to be arranged in a retracted position, in which the respective stop 80 does not reach into the groove 720 from the outside, so that further movement of the traveler 76 and the outer sheath 10/capsule 40 along the longitudinal axis x in the proximal direction P is allowed for fully deploying the implant when both stops 80 are arranged in their retracted position.

As illustrated in FIGS. 11A to 11D, for moving the respective stop 80 between the advanced and the retracted position, each stop 80 can be operatively connected to the associated manually operable actuating element 81. These elements 81 are arranged on the deployment knob 72, preferably on opposite sides. This allows actuating these elements 81 using a thumb and an index finder of the same hand.

Particularly, the actuating elements 81 can be designed as pivotable actuating elements 81 that, as illustrated in FIGS. 11A to 11D, each include a first end section 81a to which the associated stop 80 is connected, a middle section 81b that is pivotably supported on the deployment knob 72, and a second end section 81c that is configured to be pushed against the action of a spring 82 to move the respective stop 80 from its advanced position to its retracted position. The respective spring 82 can be arranged between a wall section 722 of the deployment knob 72 and the second end section 81c, 85c of the respective actuating element 81, 85. Particularly, each actuating element 81, 85 is arranged in an associated recess 723 formed in an outside 724 of the deployment knob 72. Particularly, these recesses 723 face away from each other.

If the physician is not satisfied with the position of the prosthesis and the re-sheathing limit has not been passed after actuation of the actuating elements 81 the physician can move the outer sheath 10 back in the distal direction D and therefore position the implant 100 again below the capsule 40. This movement is exactly the same as the deployment movement; just the direction of rotation is inverted (i.e. R2″).

In order to improve handling of the handle shown in FIG. 1, the deflection knob 74, the axial positioning knob 73, and the deployment knob 72 each include a surface structure 74c, 73c, 72c, wherein each surface structure 74c, 73c, 72c differs from the other surface structures, so that each knob 72, 73, 74 of the group included of the deflection knob 74, the axial positioning knob 73, and the deployment knob 72 can be identified by a physician upon touching the respective knob 72, 73, 74.

Furthermore, to also provide a visual aid, each knob of the group included of the deflection knob 74, the axial positioning knob 73, and the deployment knob 72 can include at least a first indicative (e.g. graphical) symbol 74d, 73d, 72d indicating an adjustment of a function of the catheter device 1 associated with the first rotation direction R1, R1′, R1″ of the respective knob 72, 73, 74. The axial positioning of the capsule 40 by the axial positioning knob 73, i.e., the simultaneous movement of the inner and outer sheath 20, 10 along the longitudinal axis x, is also denoted as axial fine positioning (AFP) in contrast to advancing or retracting the whole catheter by moving the handle 70. AFP enables to precisely control the height of the medical implant at the target site. Particularly, regarding this axial movement, the user needs to know how much of the travel has been used and how much more is available.

According to an embodiment shown in FIG. 1, the catheter device 1 includes an axial fine positioning indicator 790 including a scale marking 72e arranged on an outside 72g of the distal end section 72a of the deployment knob 72, and a proximal end 71b of the grip portion 71 of the handle 70, wherein the scale marking 72e moves with respect to the proximal end 71b of the grip portion 71 when the axial positioning knob 73 is rotated in the first or second rotation direction R1′, R2′ of the axial positioning knob 73, so that a current amount of a simultaneous travel of the inner and the outer sheath 20, 10 along the longitudinal axis x can be inferred from the position of the scale marking 72e with respect to the proximal end 71b of the grip portion 71. The fact that the scale marking 72e moves with respect to said edge 71b of the grip portion 71 of the handle 70 is due to the fact that rotating the axial positioning knob 73 moves the handle core 75 (cf. e.g. FIG. 7A) on which the deployment knob 72 is rotatably supported.

FIG. 12 indicates the position of the scale marking 72e of FIG. 1 in comparison to alternative axial fine positioning indicators 790. Particularly, instead of using the distal end section 72a of the deployment knob 72, the scale marking 72e′ may also be provided on the outside 751 of the handle core 75 (cf. also FIG. 8), particularly in a region between the deployment knob 72 and the axial positioning knob 73. Here, the axial fine positioning indicator 790 would further include a distal end 73a of the deployment knob 73 in relation to which the handle core's 75 scale marking 72e′ is configured to indicate a current amount of the simultaneous travel of the inner and the outer sheath 20, 10 along the longitudinal axis x. It is to be noted that the scale marking 72e′ is not visible in FIG. 12 due to the fact that the deployment knob 72 is arranged adjacent the axial positioning knob 73 in FIG. 12. However, in case the axial positioning knob 73 is rotated in its first rotation direction R1′ the handle core 75 and therewith the deployment knob 72 moves in the distal direction D (cf. also FIG. 7A) so that the marking 72e′ becomes visible.

Furthermore, alternatively, as also indicated in FIG. 12, the scale marking 72e″ of the axial fine positioning indicator 790 may also be provided (e.g. printed) on the outside 75c of the proximal end section 75b of the handle core 75, wherein here the indicator 790 can include a proximal end 74a of the deflection knob 74 in relation to which the scale marking 72e″ on the proximal end section 75b of the handle core 75 would indicate the simultaneous travel of the inner and the outer sheath 20, 10.

According to a further embodiment, the handle 70 includes a deflection indicator 791 for indicating a deflection of the deflection sheath 60 and therewith an angular orientation of the medical implant 100.

Particularly, the shape, particularly curvature, of the catheter device 1 is steered with help of the deflection sheath 60 and deflection knob 74. Particularly, by rotating the deflection knob 74, the pulling member (e.g. pull wire) 62 can be tensioned so as to bend the deflection sheath 60 which results in adjusting said angular orientation of the medical implant 100. Preferably. the user needs to know what the current angular orientation is like. For example, the user has to deactivate the deflection function before or during the withdrawal of the catheter device from the aortic arch.

Particularly, as shown in FIG. 13 the deflection indicator 791 can use the fact that the deflection can be linked to the displacement of the anchor 77 to which the pulling member 62 is connected (cf. FIGS. 3 and 6). Particularly, the anchor 77 is tensioning the pulling member 62 by moving in proximal direction P when the deflection knob 74 is rotated in the first rotation direction R1.

Therefore, as further shown in FIG. 13, the deflection indicator 791 may include an indicating member 77a that is connected to the anchor 77 and configured to be moved along a scale marking 78e included by the deflection indicator 791, which scale marking 78e of the deflection indicator 791 can be arranged on an outside 78f of the support portion 78 of the handle 70. Particularly, the support portion 78 can be at least partially transparent so that the indicating member 77a is visible from the outside below the scale marking 78e when the anchor 77 is moving due to rotating the deflection knob 74.

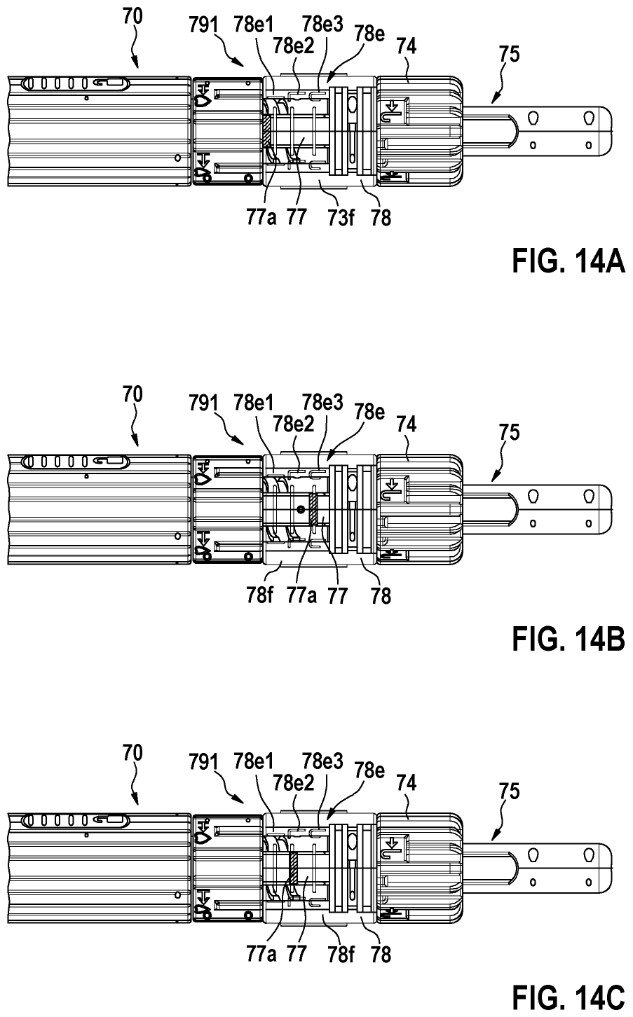

In this regard, FIGS. 14A to 14C show different deflection states indicated by the indicating member 77a with respect to the scale marking 78e.

As shown in FIGS. 13, and 14A to 14C, the scale marking 78e includes symbols arranged on the transparent support portion 78 that indicate the different states: A first symbol (e.g. in form of a straight line) 78e1 indicates that no deflection is applied to the deflection sheath 60, a second symbol (e.g. in form of a 90° curved line) 78e2 indicates a pre-deflection of the deflection sheath 60 to cross the aortic arch, and a third symbol (e.g. in form of a 180° “candy cane” line) 78e3 indicates a fine adjustment for the final placement of the implant 100. The deflection knob 74 can include corresponding symbols 74d to indicate the rotation direction towards the 180° deflection of the deflection sheath 60 as shown in FIG. 1.

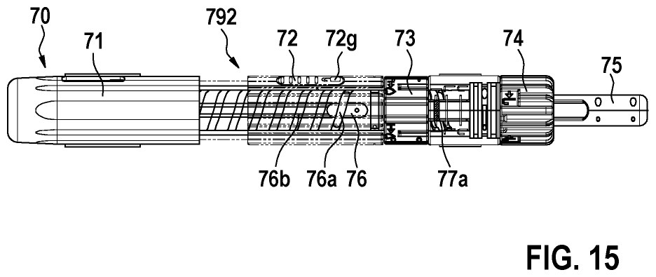

Furthermore, according to an embodiment shown in FIG. 15, the handle 70 of the catheter device 1 can also include a deployment indicator 792 that is configured to indicate an axial position of the outer sheath 10 with respect to the longitudinal axis x regarding the movement of the outer sheath 10 that can be generated by rotating the deployment knob 72.

This axial position (i.e. the deployment state) can be linked to the movement of the traveler 76, since the traveler 76 takes along the outer sheath 10 and therewith the capsule 40 when the deployment knob 72 is rotated (cf. also FIG. 8A). Particularly, the deployment indicator 792 can include an indicating member 76a that is formed by the traveler 76 (or connected to the traveler 76 or arranged on the traveler 76) and configured to be moved along a scale marking 76b included by the deployment indicator 792, which scale marking 76b of the deployment indicator 792 can be arranged on the deployment knob 72, particularly on the outside 72g of the deployment knob 72. Particularly, the deployment knob 72 can be at least partially transparent, so that the indicating member 76a is visible from the outside below the scale marking 76b when the traveler 76 is moving due to rotating the deployment knob 72 which is illustrated in FIG. 15.

The afore-described indicators 790, 791, 792 of the handle 70 allow visualizing the use of the AFP, deflection and deployment on the handle 70 in an advantageous manner.

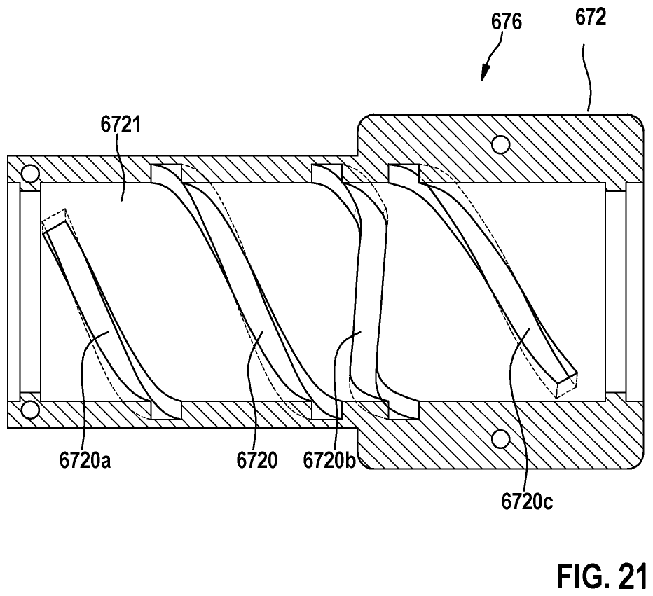

Another aspect of the present invention allows indicating to a user the point of no return after which a resheathing of an implant 6100 by a catheter device 601 for delivering the implant 6100 is no longer possible. According to the embodiments shown in FIGS. 21 and 607 this can either be accomplished by providing a deployment knob 672 on a handle 670 of a catheter device 601 with a helical groove 6720 for driving a traveler 676 connected to an outer sheath 610 of the catheter device 601 with a section 6720b having a reduced pitch to idle the traveler 676 upon rotation of the handle's 670 deployment knob 672 (cf. e.g. FIG. 21). Alternatively, the handle 670 can be configured to move at least a first protrusion 6760 of the traveler 676 out of the helical groove 6720.

These principle embodiments will be described below in more detail in context with the functions of the handle 670 and other components of the catheter device 610.

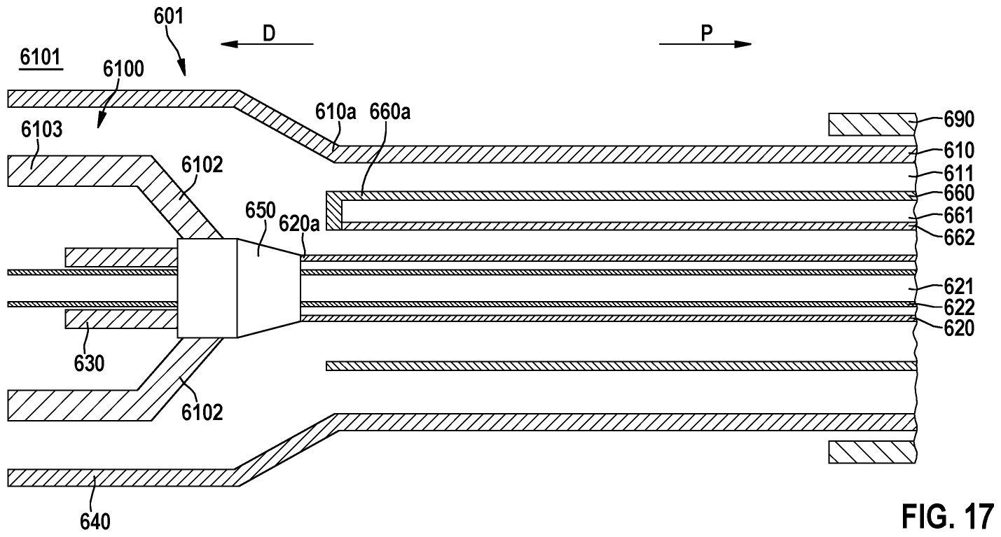

The handle 670 is particularly adapted to be used for a steerable triaxle catheter device 601 which positions a medical implant 6100, particularly a prosthetic heart valve prosthesis such as a prosthetic aortic heart valve that includes a self-expanding stent 6101 with a tissue-based valve that can include three valve leaflets connected to the stent 6101 that is schematically indicated in FIG. 17 for example in a crimped state, wherein the leaflets (not shown in FIG. 17) can be made out of a biological tissue. The stent 6101 preferably includes a plurality of interconnected struts 6103, so that the stent 6101 forms a circumferential scaffold including a plurality of lateral openings. The prosthetic heart valve 6100 is particularly designed to replace a native aortic valve. The catheter device 601 can facilitate prosthesis delivery as well as axial positioning and angular orienting. Furthermore, the handle 670 can enable partial deployment, full deployment and recapturing (resheathing) of the medical implant/prosthetic heart valve 6100.

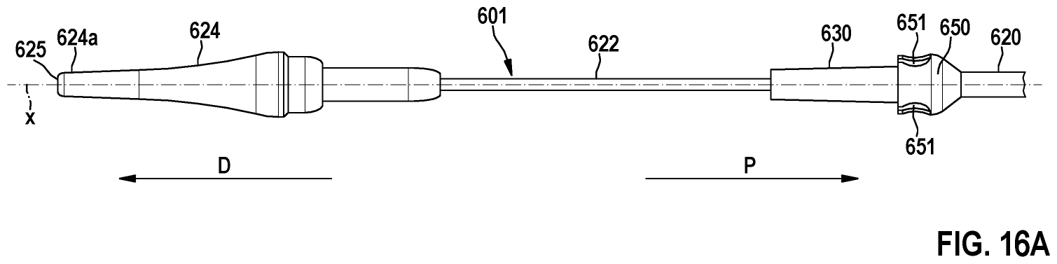

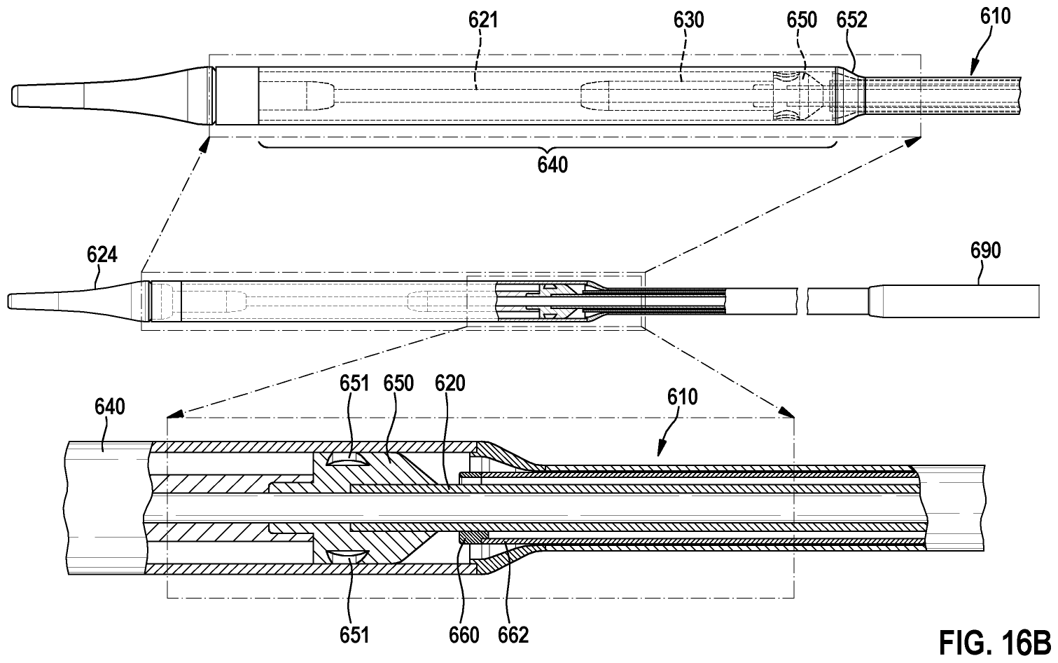

Particularly, the handle 670 is adapted to manipulate, particularly steer, four sheaths 620, 610, 660, and 690 (cf. e.g. FIGS. 16A, 16B, and 17) for example, namely: an inner sheath 620 (also denoted as inner shaft 620) enclosing a guidewire lumen 621, a deflection sheath 660 (also denoted as deflection shaft 660) enclosing the inner sheath 620, an outer sheath 610 (also denoted as outer shaft 610) enclosing the inner sheath 620 and the deflection sheath 660, as well as a stabilizing sheath 690 (also denoted as stabilizing shaft or short “stabilizer” 690) enclosing a proximal section of the inner sheath 620, deflection sheath 660 and outer sheath 610. Particularly, each sheath 610, 620, 660 forms a tubular member.

As indicated in FIG. 17, the deflection sheath 660 can be deflected with a pulling member such as a pull wire 662 (for example a stainless-steel wire) that can be connected to a distal end section 660a of the deflection sheath 660. The pull wire 662 is arranged in a lumen of the deflection sheath 660.

The deflection sheath 660 can include lateral openings so that the deflection sheath can be deflected by tensioning the pull wire 662. Particularly, in this way, the deflection sheath 660 can be deflected beyond 180°. In the letter case, the deflection sheath includes a u-shaped distal end section 660a.

The inner and the outer sheath 620, 610 can be moved relative to the deflection sheath 660 (and relative to the stabilizing sheath 690). Additionally, the outer sheath 610 can be moved relative to the inner sheath 620. The stabilizing sheath 690 is stationary and fixed to a grip portion 671 of the handle 670. The stabilizing sheath 690 does only bridge the movements of the other sheaths 610, 620, 660 to the anatomy of the patient and/or an introducer, if necessary. Particularly, the deflection sheath 660 is shorter than the inner and the outer sheath 620, 610. Further, a capsule 640 can be connected to a distal end 610a of the outer sheath 610 (e.g. via a capsule connector 652, cf. FIG. 16B), wherein the capsule 640 can be larger in diameter (perpendicular to the longitudinal axis x along which the sheaths 610, 620, 660, 690 extend) than a proximal section of the outer sheath 610.

For delivery using the catheter device 601, the medical implant 6100, here an aortic prosthetic heart valve 6100, is placed on a support element 630 that is connected to a distal end section 620a of the inner sheath 620 and covered by the capsule 640. The support element 630 can be connected to the inner sheath 620 via a connector 650 to which the heart valve prosthesis 6100 is releasably connectable for delivery to an implantation site when it is arranged in the capsule 640. The guidewire lumen 621 can be formed by a tubing 622 that protrudes out of the inner sheath 620 at the distal end 620a of the inner sheath 620, extends through the connector 650 and support element 630, and connects to a catheter tip 624 to which the tubing 622 is connected. Furthermore, the catheter tip 624 can include an opening 625 formed in a distal end 624a of the catheter tip 624, so that the guidewire can exit the guidewire lumen 621 via said opening 625 (cf. FIGS. 16A and 17).

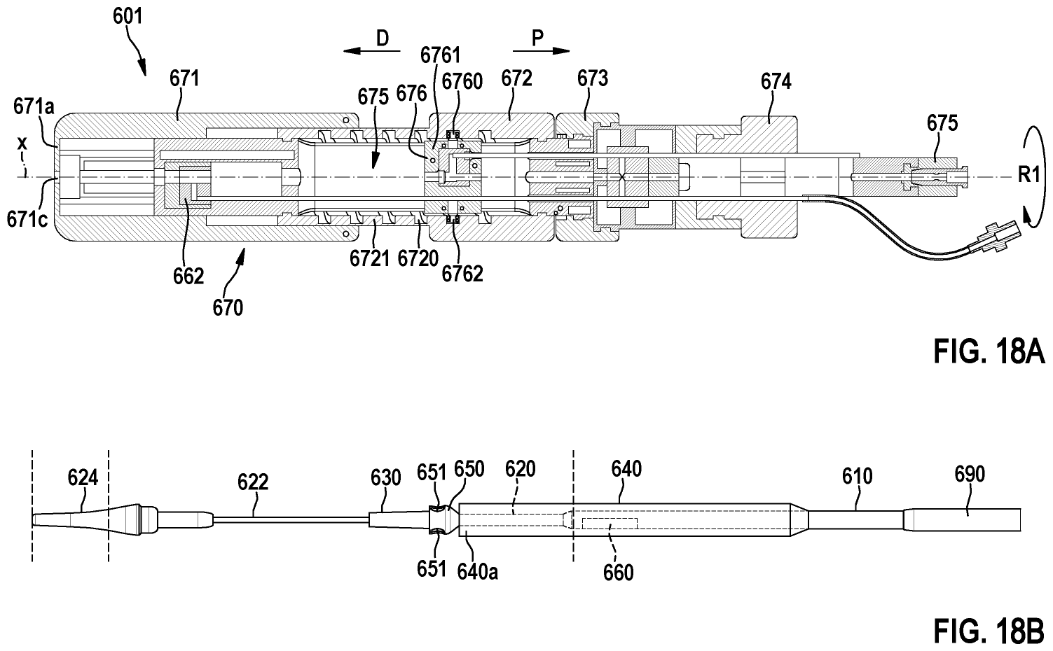

The movements and functions of the catheter device 601 can be realized with the handle 670 of the catheter device 601, wherein an embodiment of the handle 670 is shown in FIG. 18A. To this end, the handle 670 includes:
a stationary grip portion 671 configured for manually holding the handle 670 and to move the entire catheter device 601;
a rotatable deployment knob 672 for deploying and releasing the implant 6100 by moving the outer sheath 610 in a proximal direction P with respect to the inner sheath 620 and the implant 6100.

The handle 670 can further include actuating means for achieving a simultaneous movement of the inner and outer sheath 620, 610 as well as for deflecting the sheaths 610, 620 using the deflection sheath 660 and pull wire 662.

Preferably, the knob 672 is rotatable about the longitudinal axis x of the catheter device 601, along which axis x the handle 670 extends.

Furthermore, the grip portion 671 includes an opening 671c at a distal end 671a of the grip portion 671 through which all sheaths 610, 620, 660, 690 extend into the handle 670 at the distal end 671a of the grip portion 671.

For deflecting the deflection sheath 660, an actuating means such as a deflection knob 674 provided on the handle 670 can operatively connected to a distal end section 660a of the deflection sheath 660 via the pull wire 662, so that the deflection sheath 660 and thereby the inner and the outer sheath 620, 610 are deflected, i.e. bent, to adjust an angular orientation of the medical implant 6100 when the deflection knob 674 is rotated about the longitudinal axis x which causes tensioning or loosening of the pull wire 662 depending on the direction of the rotation.

Further, for moving the inner and the outer sheath 620, 610 simultaneously, the handle 670 can include a handle core 675 (cf. FIG. 18A) that is movable with respect to the grip portion 671 along the longitudinal axis x, wherein the inner and the outer sheath 620, 610 are connected to the handle core 675, and an actuating means such as an axial positioning knob 673 can be operatively connected to the handle core 675 such that the inner and the outer sheath 620, 610 are simultaneously moved with the handle core 675 with respect to the grip portion 671 (and with respect to the deflection sheath 660 and stabilizing sheath 690) along the longitudinal axis x when the axial positioning knob 673 is rotated about the longitudinal axis x.

Further, the handle 670 includes a traveler 676 (also denoted as outer sheath hub), wherein the outer sheath 610 is connected to the traveler 676, and wherein the deployment knob 672 is operatively connected to the traveler 676 such that the traveler 676 and thereby the outer sheath 610 are moved along the longitudinal axis x with respect to the inner sheath 620, and so as to deploy the medical implant 6100 when the deployment knob 672 is rotated in a first rotation direction of the deployment knob 672.

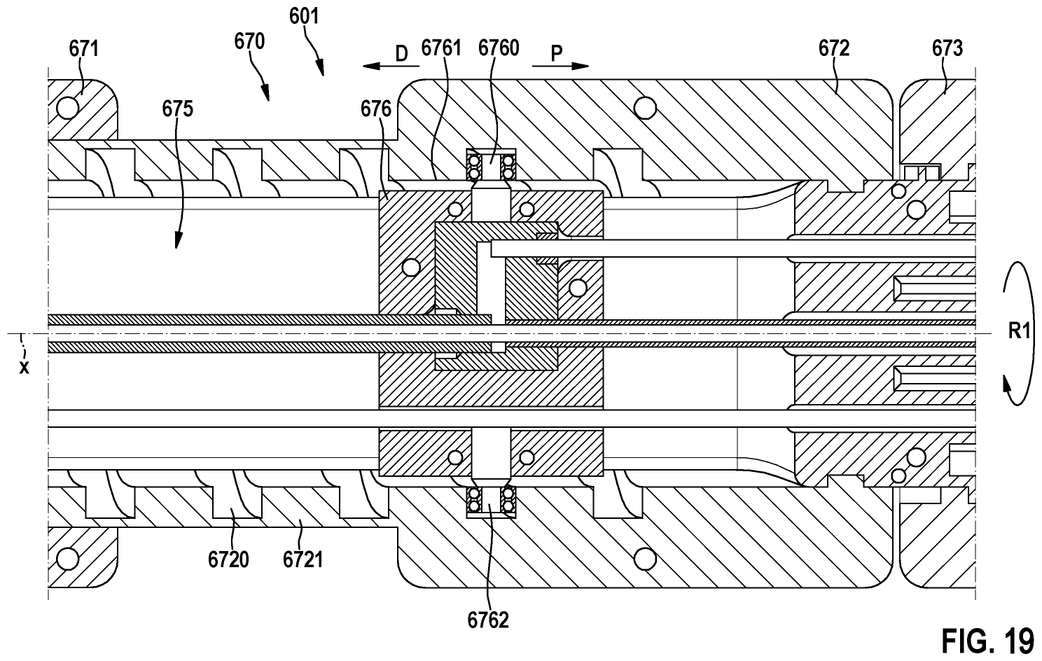

If the physician is satisfied with the positioning of the catheter device 601, deployment of the prosthesis 6100 is started by the physician. In this regard, an axial movement of the outer sheath 610 (and therewith of the capsule 640 and implant 6100) in the proximal direction P relative to all other sheaths 620, 660, 690 and the grip portion 671 of handle 670 releases the implant 6100; e.g. a prosthetic aortic heart valve. This movement of the outer sheath 610 can be controlled by the deployment knob 672 (cf. FIGS. 18 and 19), wherein rotation of the deployment knob 672 moves the traveler 676 to which the outer sheath 610 is connected. The movement of the outer sheath 610 and therewith of the capsule 640 in the proximal direction P releases the implant 6100.

For releasing the implant 6100 based on the above-described movement of the outer sheath 610 and capsule 640, the connector 650 connected to a distal end 620a of the inner sheath 620 includes a recess 651, wherein the at least one fastening element 102 of the medical implant 6100 is engaged with the at least one recess 651 as long as the capsule 640 covers the at least one recess 651 of the connector 650 and the at least one fastening element 6102 that engages with the at least one recess 651 when the prosthetic heart valve 6100 is arranged on the support 630 (cf. FIGS. 16A, 16B and 17).

The at least one fastening element 6102 can be formed by a portion of the self-expandable stent 6101 of the prosthetic heart valve 6100. Particularly, as shown in FIG. 17, the at least one fastening element 6102 can be connected to at least one strut 6103 of the stent 6101 at a proximal end of the stent 6101. For example, the stent 6101 can include three such fastening elements 6102. In this case, the connector 650 includes three corresponding recesses 651.

Once the capsule 640 is completely removed from the implant 6100 and does no longer cover the at least one fastening element 6102 and the corresponding recess 651 of the connector 650 the at least one fastening element 6102 disengages with the connector 650 due to the self-expanding property of the implant 6100/stent 6101, which releases the implant 6100 at the implantation site. After complete deployment and release of the implant 6100 from the catheter device 601, resheathing of the implant 6100 is no longer possible.

The invention therefore provides a means for indicating to the user that such a complete deployment and release (point of no return) is imminent when the user rotates the deployment knob 672 in the first rotation direction R1.

To this end, according to the embodiment shown in FIGS. 18, 19 and 21 the helical groove 6720 includes a section 6720b having a pitch that is significantly smaller, particularly zero, in comparison to the pitch of neighboring sections 6720a, 6720c on both sides of this section 6720b.

Particularly, for operatively connecting the deployment knob 672 with the traveler 676, the deployment knob 672 is rotatably supported e.g. on the handle core 675 and includes a helical groove 6720 formed in a circumferential inside 6721 of the deployment knob 672 as shown in FIG. 21, wherein the traveler 676 includes at least a first protrusion 6760 and preferably a second protrusion 6762 each protruding from an outside 6761 of the traveler 676 (cf. FIGS. 18 to 20), respectively, wherein the protrusions 6760, 6762 engage with the helical groove 6720 of the deployment knob 672 such that the traveler 676 and thereby the outer sheath 610 are moved in a proximal direction P along the longitudinal axis x with respect to the inner sheath 620 when the deployment knob 672 is rotated in the first rotation direction R1 of the deployment knob 672, so that the capsule 640 is pulled away from the medical implant 6100 in the proximal direction P to deploy the medical implant 6100.

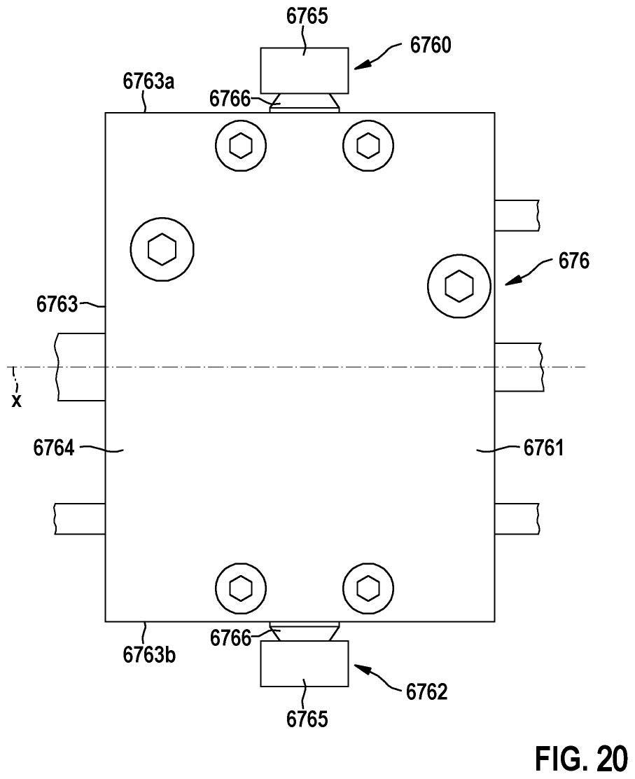

Particularly, as shown in FIG. 20, the traveler 676 includes a body 6763 having a top surface 6763a that forms a portion of the outside 6761 of the traveler 676 and a bottom surface 6763b that forms a portion of the outside 6761 of the traveler 676, too, wherein the bottom surface 6763b faces away from the top surface 6763a.

As further indicated in FIG. 20, the first protrusion 6760 of the traveler 676 preferably protrudes from the top surface 6763a while the second protrusion 6762 preferably protrudes from the bottom surface 6763b in the opposite direction. Particularly, each protrusion 6760, 6762 can include a bearing 6765 that can be supported on a pin 6766 protruding from the body 6763 of the traveler 676, wherein the respective bearing 6765 is rotatable about a rotation axis that extends perpendicular to the longitudinal axis x for reducing friction when the projections 6760, 6762 engage into the helical groove 6720 of the deployment knob 672 via the bearings 6765. Alternatively, one may also use protrusions 6760, 6762 in the form of pins, i.e., without bearings thereon.

As further indicated in FIG. 20, the body 6763 of the traveler 676 includes a through-hole 6764 extending along the longitudinal axis x of the catheter device 601 through the body 6764 of the traveler 676. The through-hole 6764 serves for fixing the outer sheath 6610 to the traveler 676. To this end, the outer sheath 610 includes a proximal end section that is arranged in the through-hole 6764 and connected to the body 6763 of the traveler 676. Particularly, the sheaths 6620, 6660 and tubing 622 inside the outer sheath 610 pass all the way through the through-hole 6764.

Now, in order to provide haptic feedback to the user, said section 6720b of the helical groove 6720 introduced above is positioned such on the inside 6721 of the deployment knob 672 that the protrusions 6760, 6762 move into this section 6720b immediately before the complete deployment and release of the implant 6100 takes place, wherein due to the reduced pitch, the deployment knob 672 idles and the traveler 676 does no longer move as long as the protrusions 6760, 6762 of the traveler 676 engage this section 6720b of the helical groove 6720 so that the user feels a pronounced lack of resistance which signals that further rotation of the deployment knob 672 will result in the complete deployment and release of the implant 6100 from the catheter device 601. Particularly, as indicated in FIG. 21, the pitch of the section 6720b of the helical groove 6720 can be adapted such that this section 6720b of the helical groove extends in the circumferential direction of the deployment knob 672 (i.e. in a plane perpendicular to the longitudinal axis x).

In other words, the pitch of said section 6720b of the helical groove of the deployment knob 672 is preferably adapted such that the helical groove 6720 is reduced to a “neutral gear”. The rotation of the knob 672 does then not move the traveler 676 for a distinct fraction of a full rotation of the knob, wherein a preferred range corresponds to 10% to 75% of a full rotation of the knob 672, particularly 25% if a full rotation as shown in FIG. 21. Particularly, if the user is satisfied with the position of the implant 6100, the user can continue to rotate the knob 672. Once the stall zone defined by section 6720b is passed, the helical groove 6720 particularly continues as before the stall zone or with an alternative pitch that results in further movement and eventually deployment and release of the implant 6100.

If the physician is not satisfied with the position of the prosthesis 6100 and the above-described re-sheathing limit (point of no return), e.g. said stall zone, has not been passed yet, the physician can move the outer sheath 610 back in the distal direction D and therefore position the implant 6100 again below the capsule 640. This movement is exactly the same as the deployment movement; just the direction of rotation of the deployment knob 672 is inverted.

Instead of using a variable pitch of the helical groove 6720, it is also possible to move the at least one first protrusion 6760 and preferably also a second protrusion 6762 of the traveler 676 (cf. e.g. FIG. 20) out of the helical groove 6720 of the deployment knob 672. According thereto, for moving the first and the second protrusion 6760, 6762 out of the helical groove 6720, the handle 672 includes a rod 680 extending along the longitudinal axis x, wherein the traveler 676 is configured to slide on said rod 680 when the deflection knob 672 is rotated in the first rotation direction R1 or in the opposite second rotation direction.

Particularly, the first protrusion 6760 is connected to a first slider 681 and the second protrusion is connected to a second slider 682, wherein each slider 681, 682 is slidably arranged in a corresponding opening 677, 678 of the body 6763 of the traveler 676 and configured to slide on a surface 680a of the rod 680 when the traveler 676 is moving along the rod 80 upon rotating the deployment knob 672.

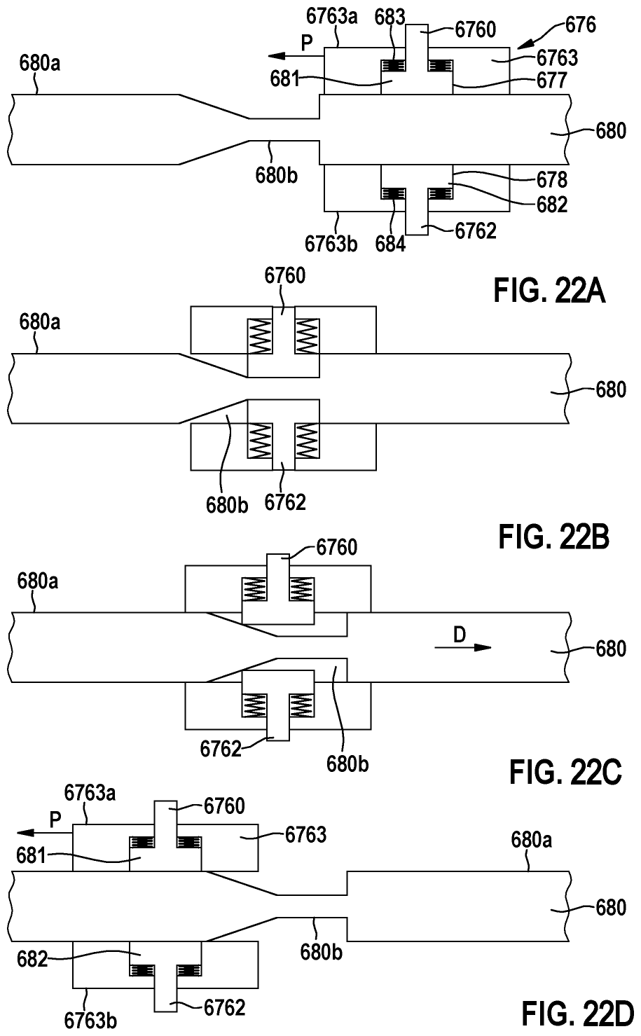

Furthermore, as indicated in FIG. 22A each slider 681, 682 is preferably pretensioned, for example by a spring 683, 684 against the surface 680a of the rod 680 such that the respective slider 681, 682 is forced to move into a recess 680b of the surface 680a of the rod 680 when the deflection knob 672 is rotated in the first rotation direction R1 and the respective slider 681, 682 moves above the recess 680b. The recess 80b is situated such that the sliders 681, 682 move into the recess 680b when a complete deployment and release of the implant 6100 is imminent. Once the sliders 681, 682 have been pressed into the recess 680b due to the force of the respective spring 683, 684 (FIG. 22B), the projections 6760, 6762 move out of the helical groove 6720 (the projections 6760, 6762 are now flush with the respective surface 6763a, 6763b of the body 763 of the traveler 676) and further movement of the traveler 676 and therewith of the outer sheath 610 in the proximal direction P when the deployment knob 672 is rotated in the first rotation direction R1 is prevented which clearly indicates the point of no return regarding resheathing of the implant 6100.

In order to reengage the protrusions 6760, 6762 with the helical groove 6720 (cf. e.g. FIG. 19) the rod 680 is for example configured to be moved in the distal direction D to push the sliders 681, 682 out of the recess 680b of the rod 680 and to thereby cause reengagement of the two protrusions 6760, 6762 of the traveler 676 into the helical groove 6720 so that further rotation of the deployment knob 672 in the first rotation direction R1 causes complete deployment and release of the implant 6100 from the catheter device 601. For pushing the sliders 681, 682 out of the recess 680b, the recess 680b can include a slope so that the respective slider is continuously lifted out of the recess 680b when the rod 680 is moved in the distal direction D by the user (FIGS. 22C and 22D).

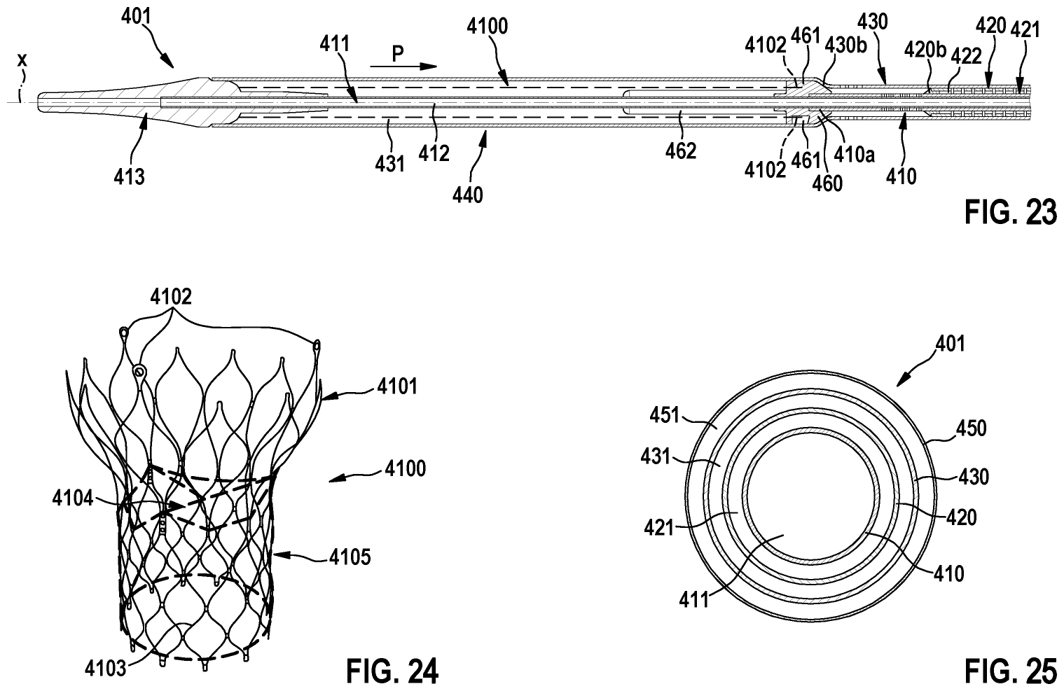

FIG. 23 shows in conjunction with FIGS. 24 to 27 an embodiment of a catheter device 401 for implanting a medical implant 4100 (cf. e.g. FIG. 24), wherein the catheter device 401 includes an inner sheath 410 extending along a longitudinal axis x and surrounding a first lumen 411 (also denoted as guidewire lumen) for receiving a guidewire, a deflection sheath 420 extending along the longitudinal axis x and surrounding a second lumen 421, wherein the inner sheath 410 extends through the second lumen 421. Furthermore, the catheter device 401 includes an outer sheath 430 extending along the longitudinal axis x, a capsule 440 connected to a distal end 430b of the outer sheath 430 for covering the medical implant 4100, wherein the outer sheath 430 and the capsule 440 together surround a third lumen 431, wherein the deflection sheath 420 is arranged in the third lumen 431. Further, the catheter device 401 includes a stabilizing sheath 450 extending along the longitudinal axis x and surrounding a fourth lumen 451, wherein the outer sheath 430 extends through the fourth lumen 431.

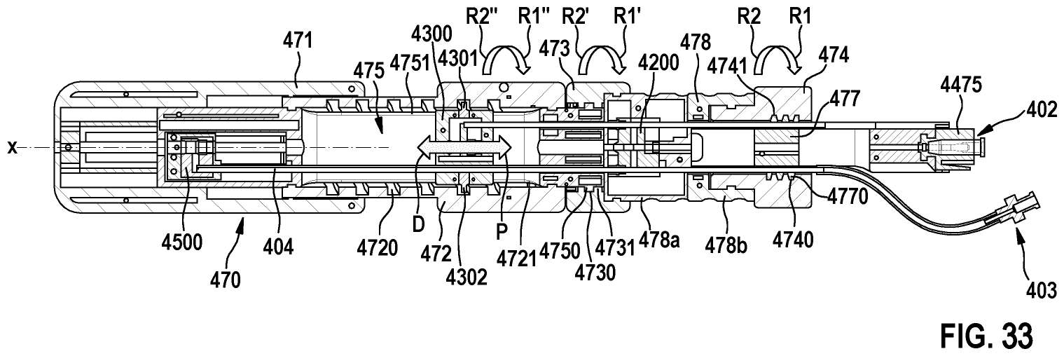

In order to control the outer, inner and stabilizing sheaths 430, 410, 420, the catheter device 401 includes a handle 470 (cf. FIG. 33) configured to move the outer sheath 430 and therewith the capsule 440 with respect to the inner sheath 410, the deflection sheath 420, and the stabilizing sheath 450 to release the medical implant 4100, wherein the handle 70 includes a first flushing port 402 being in flow connection with the first lumen 411 so as to allow flushing the first lumen 411 with a liquid medium M′ through the first flushing port 402, and wherein the handle 470 includes a second flushing port 403 being in flow connection with the fourth lumen 451, the second lumen 421, and the third lumen 431 so as to allow flushing the fourth lumen 451, the second lumen 421, and the third lumen 431 with a liquid medium M through the second flushing port 403.

The catheter device 401 can be used to transport a medical implant 4100, particularly a prosthetic heart valve prosthesis such as a prosthetic aortic heart valve to an implantation site, wherein the implant 4100 can include a self-expanding stent 4101 as shown in FIG. 24 for carrying a tissue-based valve 4104 (schematically indicated in FIG. 24) that can include three leaflets connected to the stent 4101, wherein the valve 4104 can be made out of a biological tissue. The implant 4100 can further include a circumferential skirt 4105 (schematically indicated in FIG. 24) connected to the valve 4104 and fastened to the stent 4101 for preventing paravalvular leakages. The stent 4101 preferably includes a plurality of interconnected struts 4103, so that the stent 4101 forms a circumferential scaffold including a plurality of lateral openings. The prosthetic heart valve 4100 is particularly designed to replace a native aortic valve. The catheter device 401 according to the present invention can facilitate prosthesis delivery as well as axial positioning and angular orienting. Furthermore, the handle 470 enables partial deployment, full deployment and recapturing (resheathing) of the medical implant/prosthetic heart valve.

Particularly, according to FIG. 24, the catheter device 401 can include a guidewire tube 12 connected to the inner sheath 410 to form a single first lumen 411 for receiving the guidewire. For delivery using the catheter device 401, the medical implant 4100 is placed on a support element 462 that is connected to a distal end section 410a of the inner sheath 410 and covered by the capsule 440. The support element 462 can be connected to the inner sheath 410 via a connector 460 to which the heart valve prosthesis 4100 is releasably connectable for delivery to an implantation site when it is arranged in the capsule 440. The guidewire tube 412 can protrude out of the inner sheath 10 at the distal end section 410a of the inner sheath 410, extends through the connector 460 and support element 462, and connects to a catheter tip 413 to which the tube 412 is connected, so that the guidewire can exit the guidewire lumen 411 via an opening of the tip 413. Particularly, the implant 4100 can include connecting elements 4102 that engage with recesses 461 of the connector 460, wherein the implant 4100 is released from the catheter device 4401 by moving the capsule 440 in the proximal direction P so that the connecting elements 4102 can move out of the recesses 461 due to the self-expandable scaffold 4101 (cf. FIGS. 23 and 24).

Particularly, for deploying the implant 4100, the catheter device 4401 enables a relative movement of the outer sheath 430 that is connected to the capsule 440 with respect to all other sheaths 410, 420, 450. In an embodiment, the inner sheath 410 can be configured to move in the opposite direction as the outer sheath 430 during deployment (so-called foreshortening compensation). Furthermore, the catheter device 4401 enables axial fine positioning, wherein the inner and the outer sheaths 410, 430 move together relative to the other sheaths 420, 450. Preferably, the catheter device 4401 ensures that the backflow from one lumen to the other is limited. Particularly, when the catheter device 401 is held with the distal tip 413 up once it is removed from a loading basin, the flow of the liquid medium (e.g. saline) within the system is inhibited after flushing.

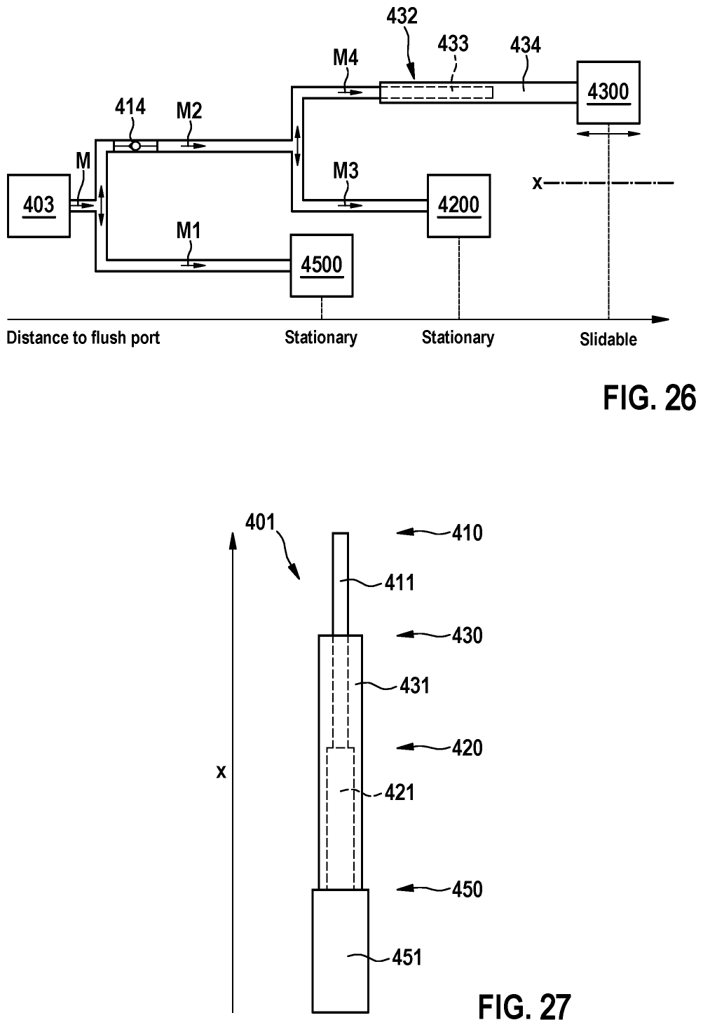

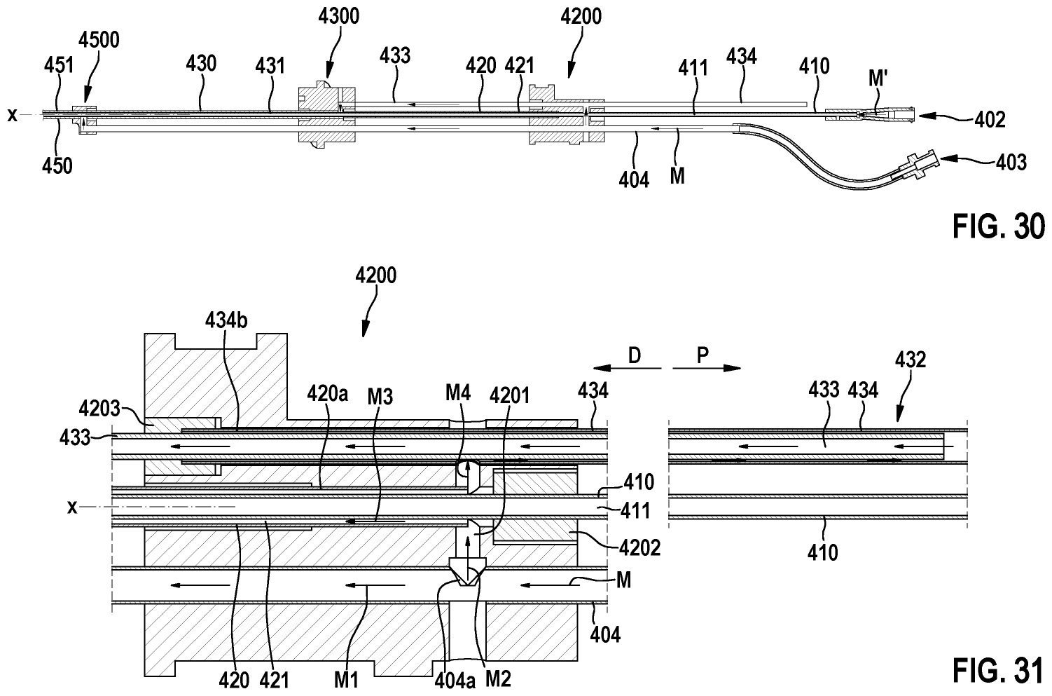

Furthermore, the first lumen 411 of the catheter device 401 can be flushed independently from the other lumens 421, 431, 451 via a first flushing port 402 (cf. FIG. 30), while the three other lumens 421, 431, 451 are configured to be flushed via a second flushing port 403 using a flushing network, wherein as shown in FIG. 26, the liquid medium/saline M is injected into the second flushing port 403 and is split in two partial streams M1, M2, wherein the first partial stream M1 is passed to a stabilizing sheath hub 4500 to which the stabilizing sheath 450 is connected to flush the fourth lumen 451, and wherein the second partial stream M2 is split again into a third and fourth partial stream M3, M4, the third partial stream M3 being passed to a deflection sheath hub 4200 to which the deflection sheath 420 is connected to flush the second lumen 421, and the fourth partial stream M4 is passed to an outer sheath hub 4300 to which the outer sheath 430 is connected to flush the third lumen 431.

Particularly, the second and fourth lumen 421, 451 do not move relative to each other. The third lumen 431/outer sheath hub 4300 however is attached by a telescopic connection 432 to the network so that the outer sheath 430/outer sheath hub 4300 can be moved relative to the rest of the flushing network. Particularly, a check valve 414 can be used to prevent liquid medium back flow from the third and the second lumen 431, 421 to the stabilizing sheath hub 4500. The check valve 414 can be installed into or close to the second flushing port 403 to avoid said backflow.

Particularly, said telescopic connection 432 includes a first and a second tube 433, 434 that are stacked so that the smaller diameter first tube 433 is always inside the larger diameter second tube 434. Preferably, the larger diameter tube 434 is stationary while the smaller diameter tube 433 is connected to the outer sheath hub 4300. The transition between the two tubes 433, 434 is preferably sealed. FIG. 26 merely shows a schematical representation of the telescopic connection. A more detailed embodiment is shown in FIG. 31 that will be described further below.

FIG. 27 shows the individual sheath exit points along the longitudinal axis x. Particularly, the order of the sheath lengths is defining how the liquid medium is moving within the system. If the catheter device 401 is held upright as shown in FIG. 27, the sheath exit points reach different heights. Particularly, the inner sheath 410 (providing the guidewire lumen) is flushed individually through the first flushing port 402 and no flow into other sheaths 420, 430, 450 is possible. Apart from the inner sheath's 410 lumen 411, the outer sheath's 30 end is most distal. Therefore, it will build up the highest fluid pressure when held upright. To keep the most distal end of the third lumen 431 flushed, the backflow from the third lumen 431 to the second lumen 421 and from the third lumen 431 to the fourth lumen 451 must be prevented. However, the deflection sheath's 420 tip is leading into the third lumen 431. Hence a backflow from the outer sheath 430 to the second sheath 420 is not possible. The backflow from the outer sheath 430 and the deflection sheath 420 to the stabilizing sheath 450 can be prevented e.g. by the check valve 414 (cf. FIG. 26).

As indicated in FIG. 30, each flushing port 402, 403 can include a Luer connector to connect to a device providing the liquid medium M (particularly saline M), e.g. a syringe.

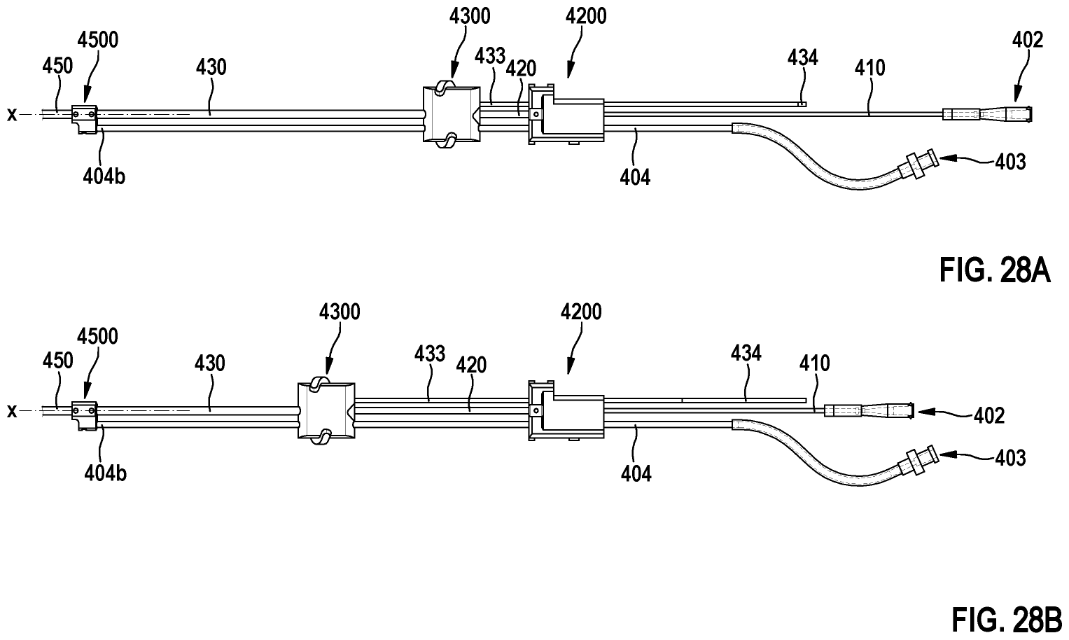

FIGS. 28A and 28B show a comparison of the positions of the stabilizing sheath hub 4500, the outer sheath hub 4300 and the deflection sheath hub 4200 during axial fine positioning upon which the capsule 440/outer sheath 430 and the inner sheath 410 are moved together along the longitudinal axis x to correctly position the implant 4100 for deployment, wherein FIG. 28A shows the most proximal position of the outer sheath hub 4300 and the inner sheath 410, and wherein FIG. 28B shows the most distal position of the outer sheath hub 4300 and the inner sheath 10. Particularly, the stabilizing sheath hub 4500 and the deflection sheath hub 4200 are stationary and do not move. Furthermore, the connection of the first flushing port 402 to the inner sheath 410 implies that the first flushing port 402 moves along the longitudinal axis x during axial fine positioning. Particularly, the deflection sheath hub 4200 and the stabilizing sheath hub 4500 are fixed to a grip portion 471 of the handle 470 of the catheter device 401 (cf. FIG. 33), whereas the outer sheath hub 4300 being connected to the outer sheath 430 is moving with respect to the deflection sheath hub 4200 and the stabilizing sheath hub 4500 during axial fine positioning.

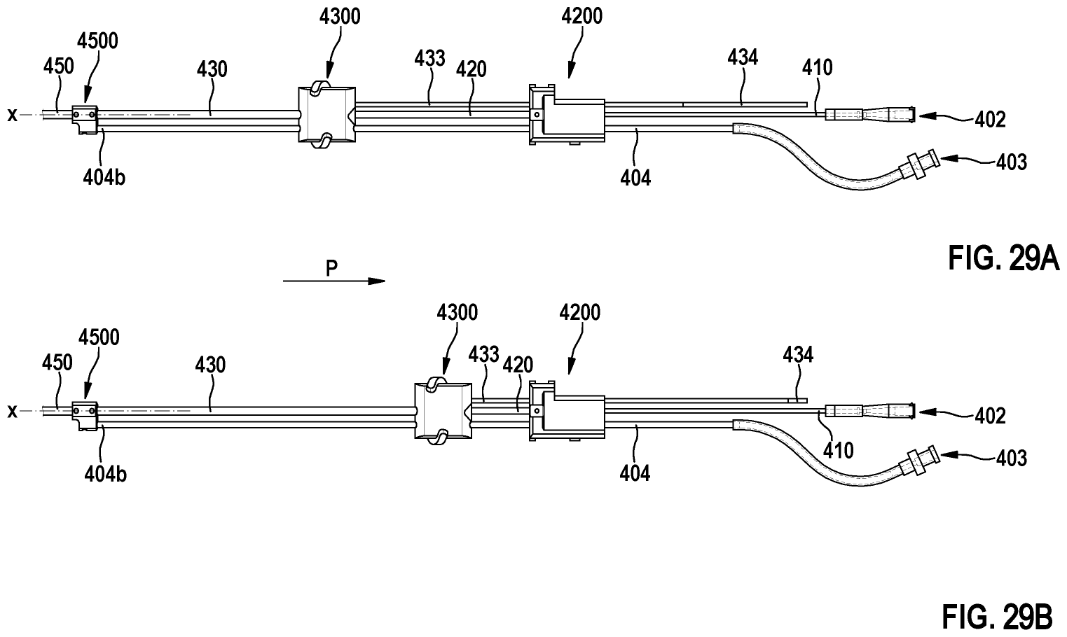

During deployment, as depicted in FIG. 29A/B, only the outer sheath hub 4300 is moving axially, i.e. along the longitudinal axis x. FIG. 29A shows the situation of the flushing network when the capsule 440 resides in its closed position, wherein the capsule 440 covers the implant 4100. In FIG. 29B, the flushing network for the opened capsule 440 is shown, i.e., the capsule 440 is retracted from the implant 4100 in the proximal direction P so that the implant 4100 automatically deploys and disengages from the connector 462.

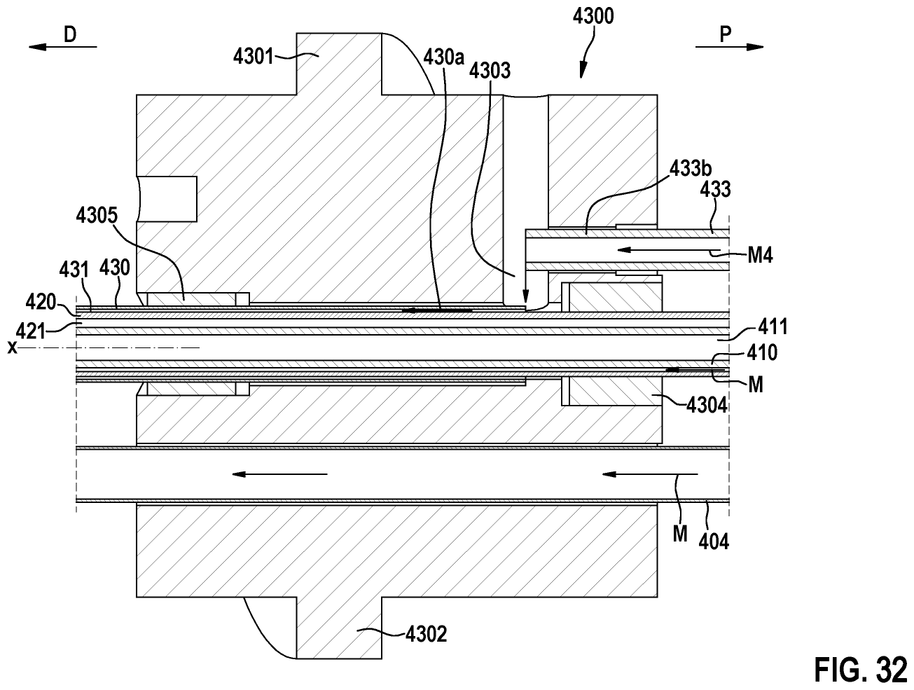

Preferably, as shown in FIG. 31, the slidable attachment of the outer sheath hub 4300 to the second flushing port 403 is realized by a telescopic connection 432, which includes a first tube 433 connected to the outer sheath hub 4300 (cf. FIG. 32) and a second tube 434 connected to the stationary deflection sheath hub 4200. When the outer sheath hub 4300 is moving, the first tube 433 slides within the second tube 434. Particularly, liquid medium M2 can enter the deflection sheath hub 200 through a lateral opening 404a of a flushing tube 4 that extends along the longitudinal axis x. The deflection sheath hub 4200 provides a flow connection 4201 between the lateral opening 404a and the second tube 434, so that liquid medium M4 can enter the second tube 434 via said flow connection 4201 and can be guided into the first tube 433 and from the first tube 433 into the outer sheath hub 4300 (cf. FIG. 32). Particularly, via said flow connection 4201, the liquid medium M4 is pushed in a gap between the two tubes 433, 434 in the proximal direction P. The second tube 434 is closed at the proximal end; hence, the liquid medium M4 is entering the first tube 433 via the proximal end of the first tube 433 and then travels in the distal direction D towards the outer sheath hub 4300.

Preferably, a junction between the tubes 433, 434 is sealed, particularly by a sealing member 4203 that can e.g. be realized by a heat shrink that is shrunk over the junction between the two tubes 433, 434. The attachment is sufficient to seal the junction but allows movement of the tubes 433, 434. According to an alternative embodiment, the sealing member 4203 can be an elastic tube (e.g. out of a silicone or another suitable material). Particularly, the sealing member 4203 seals a gap between the first tube 33 and the deflection sheath hub 4200 as well as between a distal end 434b of the second tube 34 and the deflection sheath hub 4200, so that the first tube 433 fixed to the outer sheath hub 300 can be moved with respect to the deflection sheath hub 4200 and in the second tube 434 that is fixed to the deflection sheath hub 4200. Particularly, a gap between the inner sheath 10 and the deflection sheath hub 4200 is sealed by a sealing member 4202 so that the inner sheath 410 is slidable with respect to the deflection sheath hub 4200 but said flow connection 4201 formed by the hub 4200 is sealed by this sealing member 4202.

The deflection sheath 420 is connected to the deflection sheath hub 4200 with a proximal end 420a, wherein the inner sheath 410 extends through the deflection sheath hub 4200 and is slidable along the longitudinal axis x with respect to the deflection sheath hub 4200.

Furthermore, the flushing tube 404 and the inner sheath 410 both extend through the deflection sheath hub 200 as well as through the outer sheath hub 4300, wherein no flow connection is present between the flushing tube 404 and the outer sheath hub 4300. However, a distal end 404b of the flushing tube 4 is in flow connection with the stabilizing sheath hub 4500 to pass liquid medium M1 into the fourth lumen 451 formed by the stabilizing sheath hub 4500 (cf. FIGS. 28 and 29).

FIG. 32 shows a cross-sectional view of the outer sheath hub 4300 that is movable with respect to the grip portion 471 of the handle 470 (cf. FIG. 33) to move the outer sheath 430 with respect to the inner sheath 410 as well as with respect to the deflection sheath 420 and the stabilizing sheath 450, wherein the outer sheath 430 is connected with a proximal end 30a to the outer sheath hub 4300, which proximal end 430a is connected to a distal end 433b of the first tube 433 via a flow connection 4303 provided by the outer sheath hub 4300, so that liquid medium M4 coming from the first tube 433 that is connected with its distal end 433b to the outer sheath hub 4300 can flow via said flow connection 4303 into the third lumen 431 surrounded by the outer sheath 430. A gap between the outer sheath 430 and the outer sheath hub 4300 is sealed with a sealing member 4305 to prevent the liquid medium M4 being passed through the flow connection 4303 from escaping the hub 4300 in the distal direction D.

Furthermore, the deflection sheath 420 and the inner sheath 410 extend through the outer sheath hub 4300, wherein a gap between the deflection sheath 420 and the outer sheath hub 4300 is sealed by a sealing member 4304 so that the deflection sheath 420 can slide along this sealing member 4304 and liquid medium M4 cannot escape from the outer sheath hub 4300 along the deflection sheath 420 in the proximal direction P.

As indicated in FIG. 33, the outer sheath hub 4300 as well as the inner sheath 410 can be moved using the handle 470 of the catheter device 401 in order to provide axial fine positioning of the capsule 440/implant 100 or to deploy the implant 4100. Further, using the handle 470, the deflection sheath 420 can be bent to steer the inner and outer sheaths 410, 430.

To this end, as shown in FIG. 33, the handle 470 can include a rotatable deployment knob 472, a rotatable axial positioning knob 473, and a rotatable deflection knob 474, wherein the deflection knob 474 is operatively connected to a distal end section of the deflection sheath 420, so that the deflection sheath 420 and thereby the inner and the outer sheaths 410, 430 are deflected to adjust an angular orientation of the capsule 440/implant 4100 when the deflection knob 474 is rotated in a first rotation direction R1 of the deflection knob 474, and wherein the handle 470 includes a handle core 475, wherein the inner sheath 410 is connected to the handle core 475 and wherein the outer sheath 430 is connected to the handle core 475 via the outer sheath hub 4300. Further, the axial positioning knob 473 is operatively connected to the handle core 475 such that the inner and the outer sheath 410, 430 are simultaneously moved (cf. also FIG. 28) with the handle core 475 with respect to the grip portion 471 and the deflection sheath 420 along the longitudinal axis x when the axial positioning knob 473 is rotated. Furthermore, the deployment knob 472 is operatively connected to the outer sheath hub 4300 such that the outer sheath hub 4300 and thereby the outer sheath 430 are moved along the longitudinal axis x with respect to the inner sheath 410 for deploying the medical implant 4100 (cf. also FIG. 29A/B) when the deployment knob 472 is rotated in a first rotation direction R1″ of the deployment knob 472.

Particularly, the deflection knob 474 is operatively connected to the distal end section 420b of the deflection sheath 420 via an elongated pulling member 422 (cf. FIG. 23) and via an anchor 477 of the handle 470, wherein the pulling member 422 connects the distal end section 420b of the deflection sheath 420 to the anchor 477 that is configured to be moved with respect to the grip portion 471 along the longitudinal axis x in a proximal direction P when the deflection knob 474 is rotated in the first rotation direction R1 of the deflection knob 474, such that the pulling member 422 is tensioned and the deflection sheath 420 is deflected, and wherein the anchor 477 is configured to be moved with the respect to the grip portion 471 along the longitudinal axis x in a distal direction D when the deflection knob 474 is rotated in an opposite second rotation direction R2 of the deflection knob 474 such that the pulling member 422 is loosened and a deflection of the deflection sheath 420 is reduced.

Furthermore, the deflection knob 474 includes a helical groove 4740 formed in an inside 4741 of the deflection knob 474, wherein the anchor 477 includes at least one protrusion 4770 protruding from an outside of the anchor 477, wherein the protrusion 4770 engages with the helical groove 4740 such that the anchor 477 is moved in the proximal direction P when the deflection knob 474 is rotated in the first rotation direction R1 of the deflection knob 474 and such that the anchor 477 is moved in the distal direction D when the deflection knob 474 is rotated in the second rotation direction R2 of the deflection knob 474. Particularly, the deflection knob 474 is rotatably supported on a proximal end section 78b of a support portion 478 of the handle 470, wherein the support portion 478 of the handle 470 is connected via at least one elongated connecting member to the grip portion 471 of the handle 470.

Furthermore, the axial positioning knob 473 includes a helical groove 4730 formed in an inside 4731 of the axial positioning knob 473, and wherein the handle core 475 includes a protrusion 4750 protruding from an outside 4751 of the handle core 475, wherein the protrusion 4750 of the handle core 475 engages with the helical groove 4730 of the axial positioning knob 473 to operatively connect the axial positioning knob 473 to the handle core 475 such that the handle core 475 and therewith the inner sheath 420 and the outer sheath 410 are simultaneously moved with respect to the grip portion 471 along the longitudinal axis x in the distal direction D when the axial positioning knob 473 is rotated in a first rotation direction R1′ of the axial positioning knob 473, and such that the handle core 475 and therewith the inner sheath 410 and the outer sheath 430 are simultaneously moved with respect to the grip portion 471 along the longitudinal axis x in the proximal direction P when the axial positioning knob 473 is rotated in an opposite second rotation direction R2′ of the axial positioning knob 473 so as to provide axial fine positioning of the capsule 440/implant 4100 (cf. also FIG. 28). Particularly, the axial positioning knob 473 is rotatably supported on a distal end section 478a of the support portion 478 of the handle 470.

Furthermore, for deploying the implant 4100, the deployment knob 472 is rotatably supported on the handle core 475 and includes a helical groove 4720 formed in an inside 4721 of the deployment knob 472, wherein the outer sheath hub 4300 includes two protrusions 4301, 4302 that engage with the helical groove 4720 of the deployment knob 472 such that the outer sheath hub 4300 and thereby the outer sheath 430 are moved in a proximal direction P along the longitudinal axis x with respect to the inner sheath 410 and the handle core 475 when the deployment knob 472 is rotated in the first rotation direction R1″ of the deployment knob 472, so that the capsule 440 is pulled away from the medical implant 4100 in the proximal direction P to deploy the medical implant 4100 and such that the outer sheath hub x and thereby the outer sheath 430 is moved in a distal direction D along the longitudinal axis x with respect to the inner sheath 410 when the deployment knob 472 is rotated in an opposite second rotation direction R2″ of the deployment knob 472, so that the capsule 440 is moved back over the medical implant 4100 to re-sheath a partially deployed medical implant 4100.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A catheter device for implanting a medical implant, comprising:
an outer sheath extending along a longitudinal axis of the catheter device and surrounding a lumen of the outer sheath;
an inner sheath extending along the longitudinal axis, wherein the inner sheath is arranged in the lumen of the outer sheath and connected to a support element for supporting the medical implant and/or a connector;
a capsule connected to a distal end of the outer sheath, the capsule being configured to cover the medical implant when the medical implant is arranged on the support element and/or the connector;
a handle comprising: a grip portion, f a rotatable deployment knob and a traveler, wherein the outer sheath is connected to the traveler and wherein the deployment knob is operatively connected to the traveler such that the traveler and thereby the outer sheath are moved along the longitudinal axis with respect to the inner sheath, and the handle comprises a rotatable axial positioning knob and a rotatable deflection knob; and wherein the deployment knob is arranged proximally to the axial positioning knob,
optionally a deflection sheath for adjusting the angular orientation of the capsule and/or the medical implant.

2. The catheter device according to claim 1, wherein the deployment knob is arranged adjacent the axial positioning knob, and/or wherein the rotatable axial positioning knob is situated between the rotatable deployment knob and the rotatable deflection knob, and/or wherein the grip portion is situated proximally to the deployment knob.

3. (canceled)

4. (canceled)

5. (canceled)

6. The catheter device according to claim 1,
the deflection sheath extending along the longitudinal axis and surrounding a lumen of the deflection sheath, wherein the deflection sheath is arranged in the lumen the outer sheath and the inner sheath is arranged in the lumen of the deflection sheath, and wherein the handle further comprises a rotatable axial positioning knob, and a rotatable deflection knob, wherein the deflection sheath is connected to the grip portion and the deflection knob is operatively connected to a distal end section of the deflection sheath, so that the deflection sheath and thereby the inner and the outer sheaths are deflected to adjust an angular orientation of the medical implant when the deflection knob is rotated in a first rotation direction of the deflection knob, and wherein the handle comprises a handle core, wherein the inner and the outer sheath are connected to the handle core and the axial positioning knob is operatively connected to the handle core such that the inner and the outer sheath are simultaneously moved with the handle core with respect to the grip portion and the deflection sheath along the longitudinal axis when the axial positioning knob is rotated, and wherein the deployment knob is operatively connected to the traveler such that the traveler and thereby the outer sheath are moved along the longitudinal axis with respect to the inner sheath for deploying the medical implant when the deployment knob is rotated in a first rotation direction of the deployment knob.

7. The catheter device according to claim 1, wherein the deflection knob is operatively connected to the distal end section of the deflection sheath via an elongated pulling member and via an anchor of the handle, wherein the pulling member connects the distal end section the deflection sheath to the anchor that is configured to be moved with respect to the grip portion along the longitudinal axis in a proximal direction when the deflection knob is rotated in the first rotation direction of the deflection knob, such that the pulling member is tensioned and the deflection sheath is deflected, and wherein the anchor is configured to be moved with the respect to the grip portion along the longitudinal axis in a distal direction when the deflection knob is rotated in an opposite second rotation direction of the deflection knob such that the pulling member is loosened and a deflection of the deflection sheath is reduced; and/or wherein the deflection knob comprises a helical groove formed in an inside of the deflection knob, wherein the anchor comprises a protrusion protruding from an outside of the anchor, wherein the protrusion engages with the helical groove such that the anchor is moved in the proximal direction when the deflection knob is rotated in the first rotation direction of the deflection knob and such that the anchor is moved in the distal direction when the deflection knob is rotated in the second rotation direction of the deflection knob.

8. (canceled)

9. (canceled)

10. (canceled)

11. The catheter device according to claim 1, wherein the axial positioning knob comprises a helical groove formed in an inside of the axial positioning knob, and wherein the handle core comprises a protrusion protruding from an outside of the handle core, wherein the protrusion of the handle core engages with the helical groove of the axial positioning knob to operatively connect the axial positioning knob to the handle core such that the handle core and therewith the inner sheath and the outer sheath are simultaneously moved with respect to the grip portion along the longitudinal axis in the distal direction when the axial positioning knob is rotated in a first rotation direction of the axial positioning knob, and such that the handle core and therewith the inner sheath and the outer sheath are simultaneously moved with respect to the grip portion along the longitudinal axis in the proximal direction when the axial positioning knob is rotated in an opposite second rotation direction of the axial positioning knob.

12. (canceled)

13. The catheter device according to claim 1, wherein the deployment knob is rotatably supported on the handle core and comprises a helical groove formed in an inside of the deployment knob, wherein the traveler comprises at least a first protrusion protruding from an outside of the traveler, wherein the at least one first protrusion engages with the helical groove of the deployment knob such that the traveler and thereby the outer sheath are moved in a proximal direction along the longitudinal axis with respect to the inner sheath and the handle core when the deployment knob is rotated in the first rotation direction of the deployment knob, so that the capsule is pulled away from the medical implant in the proximal direction to deploy the medical implant and such that the traveler and thereby the outer sheath is moved in a distal direction along the longitudinal axis with respect to the inner sheath when the deployment knob is rotated in an opposite second rotation direction of the deployment knob, so that the capsule is moved back over the medical implant to re-sheath a partially deployed medical implant.

14. The catheter device according to claim 13, wherein the traveler comprises a second protrusion protruding from the outside of the traveler, wherein the first and the second protrusion engage with the helical groove of the deployment knob such that the traveler and thereby the outer sheath is moved in the proximal direction along the longitudinal axis with respect to the inner sheath when the deployment knob is rotated in the first rotation direction of the deployment knob, so that the capsule is moved away from the medical implant in the proximal direction and such that the traveler and thereby the outer sheath is moved in the distal direction along the longitudinal axis with respect to the inner sheath when the deployment knob is rotated in the second rotation direction of the deployment knob, so that the capsule is moved back over the medical implant to re-sheath the partially deployed medical implant; and/or wherein the traveler comprises a body having a top surface and a bottom surface facing away from the top surface; wherein the first protrusion of the traveler protrudes from the top surface, wherein the first protrusion forms a curved wine having a first end protruding past a first lateral surface of the body and a second end protruding past a second lateral surface of the body, and/or wherein the second protrusion of the traveler protrudes from the bottom surface, wherein the second protrusion forms a curved wing having a first end protruding past the first lateral surface and a second end protruding past the second lateral surface of the body.

15. (canceled)

16. The catheter device according to claim 13, wherein for limiting a movement of the traveler in the proximal direction upon rotating the deployment knob in the first rotation direction of the deployment knob, the deployment knob comprises a first stop and a second stop, each stop configured to be arranged in an advanced position, wherein the first stop blocks the first protrusion when the first stop is arranged in its advanced position and the second stop blocks the second protrusion when the second stop is arranged in its advanced position, so that further movement of the traveler in the proximal direction is prevented and the medical implant is kept in a partially deployed state in which the medical implant is still connected to the inner shaft, and wherein each stop is configured to be arranged in a retracted position, so that further movement of the traveler and the outer sheath along the longitudinal axis in the proximal direction is allowed when both stops are arranged in their retracted position to let the medical implant assume a fully deployed state in which the medical implant is released from the inner sheath.

17. The catheter device according to claim 16, wherein the first stop is operatively connected to a manually operable first actuating element arranged on the deployment knob, wherein the first actuating element is configured to be manually operated to bring the first actuating element from a first state in which the at least one first stop is arranged in its advanced position to a second state in which the at least one first stop is arranged in its retracted position, and/or wherein the second stop is operatively connected to a manually operable second actuating element arranged on the deployment knob, wherein the second actuating element is configured to be manually operated to bring the second actuating element from a first state in which the second stop is arranged in its advanced position to a second state in which the second stop is arranged in its retracted position.

18. (canceled)

19. (canceled)

20. (canceled)

21. The catheter device according to claim 1, wherein the deployment knob comprises a helical groove formed in an inside of the deployment knob, wherein the traveler comprises at least a first protrusion that engages with the helical groove of the deployment knob such that the traveler and thereby the outer sheath are moved in a proximal direction along the longitudinal axis with respect to the inner sheath when the deployment knob is rotated in a first rotation direction, so that the capsule is pulled away from the medical implant in the proximal direction to deploy the medical implant, wherein for indicating an imminent complete deployment and release of the medical implant from the catheter device when the user rotates the deployment knob in the first rotation direction, the helical groove comprises a section having a reduced pitch or the handle is configured to move the at least one first protrusion out of the helical groove.

22. The catheter device according to claim 21, wherein the pitch is adapted such that a rotation of the deployment knob in the first rotation direction does not move the traveler and therewith the outer sheath in the proximal direction for a pre-defined fraction of a full rotation of the deployment knob in the first rotation direction when the at least one first protrusion engages with said section of the helical groove.

23. (canceled)

24. The catheter device according to claim 21, wherein the traveler comprises a second protrusion protruding from an outside of the traveler, wherein the first and the second protrusion engage with the helical groove of the deployment knob such that the traveler and thereby the outer sheath are moved in a proximal direction along the longitudinal axis with respect to the inner sheath when the deployment knob is rotated in the first rotation direction of the deployment knob, so that the capsule is pulled away from the medical implant in the proximal direction to deploy the medical implant, wherein for indicating an imminent complete deployment and release of the medical implant from the catheter device when the user rotates the deployment knob in the first rotation direction, the helical groove comprises a section having a pitch that is adapted such that a rotation of the deployment knob in the first rotation direction does not move the traveler and therewith the outer sheath in the proximal direction for a pre-defined fraction of a full rotation of the deployment knob in the first rotation direction when the at least one first protrusion and the second protrusion engage with said section of the helical groove.

25. (canceled)

26. The catheter device according to claim 21, wherein the at least one first protrusion is a first pin that is configured to engage into the helical groove of the deployment knob; and/or wherein the second protrusion is a second pin that is configured to engage into the helical groove of the deployment knob or wherein the at least one first protrusion comprises a bearing that is configured to engage into the helical groove of the deployment knob; and/or wherein the second protrusion comprises a bearing that is configured to engage into the helical groove of the deployment knob.

27. (canceled)

28. The catheter device according to claim 21, wherein for moving the at least one first protrusion out of the helical groove, the handle comprises a rod that extends along the longitudinal axis, wherein the traveler is configured to slide on said rod when the deflection knob is rotated in the first rotation direction or in the opposite second rotation direction.

29. (canceled)

30. (canceled)

31. (canceled)

32. The catheter device according to claim 1, wherein the inner sheath surrounds a first lumen for receiving a guidewire, and wherein the outer sheath and the capsule together surround a third lumen, wherein the deflection sheath is arranged in the third lumen, and wherein the catheter devices further comprises
a deflection sheath extending along the longitudinal axis and surrounding a second lumen, wherein the inner sheath extends through the second lumen,
a stabilizing sheath extending along the longitudinal axis and surrounding a fourth lumen, wherein the outer sheath extends through the fourth lumen, and wherein the handle further comprises a first flushing port being in flow connection with the first lumen so as to allow flushing the first lumen with a liquid medium through the first flushing port, and
wherein the handle further comprises a second flushing port being in flow connection with the fourth lumen, the second lumen, and the third lumen so as to allow flushing the fourth lumen, the second lumen, and the third lumen with a liquid medium through the second flushing port.

33. A catheter device for implanting a medical implant, the catheter device comprising:
an inner sheath extending along the longitudinal axis and surrounding a first lumen for receiving a guidewire,
a deflection sheath extending along the longitudinal axis and surrounding a second lumen, wherein the inner sheath extends through the second lumen,
an outer sheath extending along the longitudinal axis,
a capsule connected to a distal end of the outer sheath for covering the medical implant, wherein the outer sheath and the capsule together surround a third lumen, wherein the deflection sheath is arranged in the third lumen,
a stabilizing sheath extending along the longitudinal axis and surrounding a fourth lumen, wherein the outer sheath extends through the fourth lumen,
a handle configured to move the outer sheath with respect to the inner sheath to release the medical implant,
wherein the handle comprises a first flushing port being in flew fluid connection with the first lumen so as to allow flushing the first lumen with a liquid medium through the first flushing port, and wherein the handle comprises a second flushing port being in fluid connection with the fourth lumen, the second lumen, and the third lumen so as to allow flushing the fourth lumen, the second lumen, and the third lumen with a liquid medium through the second flushing port.

34. The catheter device according to claim 32, wherein the flow connection between the second flushing port and the fourth lumen, the second lumen, and the third lumen is configured such that a liquid medium injected into the second flushing port is split into a first and a second partial stream, the first partial stream flushing the fourth lumen, and wherein the second partial stream is split again into a third and a fourth partial stream, the third partial stream flushing the second lumen and the fourth partial stream flushing the third lumen.

35. The catheter device according to claim 32, wherein the second flushing port is in flow connection with the third lumen via a telescopic connection comprising a first tube and a second tube, wherein the first tube is configured to slide along the second tube.

36. The catheter device according to claim 32, wherein the handle comprises an outer sheath hub that is movable with respect to a grip portion of the handle to move the outer sheath with respect to the inner sheath, wherein the outer sheath is connected with a proximal end to the outer sheath hub, and wherein the deflection sheath and the inner sheath extend through the outer sheath hub or wherein the handle comprises a deflection sheath hub that is stationary with respect to the grip portion of the handle, wherein a proximal end of the deflection sheath is connected to the deflection sheath hub.

37. (canceled)

38. (canceled)

39. (canceled)

40. (canceled)

41. (canceled)

42. (canceled)

43. (canceled)

44. The catheter device according to claim 33, wherein the handle further comprises a rotatable deployment knob, a rotatable axial positioning knob, and a rotatable deflection knob, wherein the deflection knob is operatively connected to a distal end section of the deflection sheath, so that the deflection sheath and thereby the inner and the outer sheaths are deflected to adjust an angular orientation of the capsule when the deflection knob is rotated in a first rotation direction of the deflection knob, and wherein the handle comprises a handle core, wherein the inner sheath is connected to the handle core and wherein the outer sheath is connected to the handle core via the outer sheath hub, and wherein the axial positioning knob is operatively connected to the handle core such that the inner and the outer sheath are simultaneously moved with the handle core with respect to the grip portion and the deflection sheath along the longitudinal axis when the axial positioning knob is rotated, and herein the deployment knob is operatively connected to the outer sheath hub such that the outer sheath hub and thereby the outer sheath are moved along the longitudinal axis with respect to the inner sheath for deploying the medical implant when the deployment knob is rotated in a first rotation direction of the deployment knob.

45. (canceled)

46. (canceled)
